SEQUENCE LISTING (16 sequences)

SEQ ID NO: 1 - DNA, 378 bp, Artificial Sequence
  Oligonucleotide sequence
ccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 2 - DNA, 420 bp, Artificial Sequence
  Oligonucleotide sequence
attgaagcatgcggcgtgcataattctcttactgtcatgccatgcgtaagataccaccacacccgcattcgccattcaggcggccgccaccgcggtggagctccagctgctgtttcctgtgtagagttggtagctcttgatccggtcatatttgttccctttagatccgcctccatctacagggcgcgtccccgcgcttaatgcgcggcctaactacggctacactagaaggacttaccttcggaaaagaaattgttatccgctcacaaaagccagagtatttaagctccctcgtgcgctctcctgttccgggttattgtctcatcggcgaccgagttgctcttgcttatcagaccctgccgcttacaagtggtcgccagtctattaacagcactcaatacgggataatttttcaatatt

SEQ ID NO: 3 - DNA, 78 bp, Artificial Sequence
  Oligonucleotide sequence
gaaccgtcccaagcgttgcgccacatctgctggaaggtggacagtgagaggacacctacgaatcgcaacgggtatcct

SEQ ID NO: 4 - DNA, 77 bp, Artificial Sequence
  Oligonucleotide sequence
gaaccgtcccaagcgttgcgcctgggtacatggtggtaccaccagacaggacacctacgaatcgcaacgggtatcct

SEQ ID NO: 5 - DNA, 76 bp, Artificial Sequence
  Oligonucleotide sequence
gaaccgtcccaagcgttgcggagagcatagccctcgtagatgggcaaggacacctacgaatcgcaacgggtatcct

SEQ ID NO: 6 - DNA, 81 bp, Artificial Sequence
  Oligonucleotide sequence
gaaccgtcccaagcgttgcggtcccagttggtaacaatgccatgttcaatgaggacacctacgaatcgcaacgggtatcct

SEQ ID NO: 7 - DNA, 76 bp, Artificial Sequence
  Oligonucleotide sequence
gaaccgtcccaagcgttgcgcggactcatcgtactcctgcttgctgaggacacctacgaatcgcaacgggtatcct

SEQ ID NO: 8 - DNA, 78 bp, Artificial Sequence
  Oligonucleotide sequence
gaaccgtcccaagcgttgcgttctctttgatgtcacgcacgatttcccaggacacctacgaatcgcaacgggtatcct

SEQ ID NO: 9 - DNA, 80 bp, Artificial Sequence
  Oligonucleotide sequence
gaaccgtcccaagcgttgcgctcggtcaggatcttcatgaggtagtctgtaggacacctacgaatcgcaacgggtatcct

SEQ ID NO: 10 - DNA, 78 bp, Artificial Sequence
  Oligonucleotide sequence
gaaccgtcccaagcgttgcgtttcacggttggccttagggttcaggggaggacacctacgaatcgcaacgggtatcct

SEQ ID NO: 11 - DNA, 78 bp, Artificial Sequence
  Oligonucleotide sequence
gaaccgtcccaagcgttgcggtacttcagggtcaggatacctctcttgaggacacctacgaatcgcaacgggtatcct

SEQ ID NO: 12 - DNA, 79 bp, Artificial Sequence
  Oligonucleotide sequence
gaaccgtcccaagcgttgcgctgctcgaagtctagagcaacatagcacaaggacacctacgaatcgcaacgggtatcct

SEQ ID NO: 13 - DNA, 76 bp, Artificial Sequence
  Oligonucleotide sequence
gaaccgtcccaagcgttgcgcctcgtcacccacataggagtccttcaggacacctacgaatcgcaacgggtatcct

SEQ ID NO: 14 - DNA, 461 bp, Artificial Sequence
  Pseudogene sequence
ggtctcacattgcataattaacatccgcggaacgcggatgttccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcatccgcggaacgcggatgcagctcttctgcattggagacc

SEQ ID NO: 15 - DNA, 510 bp, Artificial Sequence
  Pseudogene sequence
ggtctcacattgcataaatttcatccgtgggaaccacggatgaaattgaagcatgcggcgtgcataattctcttactgtcatgccatgcgtaagataccaccacacccgcattcgccattcaggcggccgccaccgcggtggagctccagctgctgtttcctgtgtagagttggtagctcttgatccggtcatatttgttccctttagatccgcctccatctacagggcgcgtccccgcgcttaatgcgcggcctaactacggctacactagaaggacttaccttcggaaaagaaattgttatccgctcacaaaagccagagtatttaagctccctcgtgcgctctcctgttccgggttattgtctcatcggcgaccgagttgctcttgcttatcagaccctgccgcttacaagtggtcgccagtctattaacagcactcaatacgggataatttttcaatattcatccgtgggaaccacggatgaatagctcttcatgcattggagacc

SEQ ID NO: 16 - DNA, 1177 bp, Artificial Sequence
  Pseudogene sequence
ggtctcacattgcataatcttcatccgtgggaaccacggatgaagaaccgtcccaagcgttgcgccacatctgctggaaggtggacagtgagaggacacctacgaatcgcaacgggtatcctcatccgtgggaaccacggatgaggaaccgtcccaagcgttgcgcctgggtacatggtggtaccaccagacaggacacctacgaatcgcaacgggtatcctcatccgtgggaaccacggatgaggaaccgtcccaagcgttgcggagagcatagccctcgtagatgggcaaggacacctacgaatcgcaacgggtatcctcatccgtgggaaccacggatgaggaaccgtcccaagcgttgcggtcccagttggtaacaatgccatgttcaatgaggacacctacgaatcgcaacgggtatcctcatccgtgggaaccacggatgaggaaccgtcccaagcgttgcgcggactcatcgtactcctgcttgctgaggacacctacgaatcgcaacgggtatcctcatccgtgggaaccacggatgaggaaccgtcccaagcgttgcgttctctttgatgtcacgcacgatttcccaggacacctacgaatcgcaacgggtatcctcatccgtgggaaccacggatgaggaaccgtcccaagcgttgcgctcggtcaggatcttcatgaggtagtctgtaggacacctacgaatcgcaacgggtatcctcatccgtgggaaccacggatgaggaaccgtcccaagcgttgcgtttcacggttggccttagggttcaggggaggacacctacgaatcgcaacgggtatcctcatccgtgggaaccacggatgaggaaccgtcccaagcgttgcggtacttcagggtcaggatacctctcttgaggacacctacgaatcgcaacgggtatcctcatccgtgggaaccacggatgaggaaccgtcccaagcgttgcgctgctcgaagtctagagcaacatagcacaaggacacctacgaatcgcaacgggtatcctcatccgtgggaaccacggatgaggaaccgtcccaagcgttgcgcctcgtcacccacataggagtccttcaggacacctacgaatcgcaacgggtatcctcatccgtgggaaccacggatgaggagctcttcatgcattggagacc

(19) United States
(12) Patent Application Publication
(10) Pub. No.: US 2022/0049291 A1
(43) Pub. Date: Feb. 17, 2022

(54) METHOD AND PRODUCTS FOR PRODUCING FUNCTIONALISED SINGLE STRANDED OLIGONUCLEOTIDES

(71) Applicant: MOLIGO TECHNOLOGIES AB, Solna (SE)

(72) Inventors: Cosimo DUCANI, Stockholm (SE); Bjorn HOGBERG, Stockholm (SE)

(21) Appl. No.: 17/428,889

(22) PCT Filed: Feb. 5, 2020
(86) PCT No.: PCT/EP2020/052868

§ 371 Date: Aug. 5, 2021

(30) Foreign Application Priority Data
Feb. 5, 2019 (GB) 1901583.3

(51) Int. Cl. C12Q 1/6811; C12Q 1/6853; C12Q 1/6841; C12N 15/10

(52) U.S. Cl. CPC C12Q 1/6811; C12N 15/1065; C12Q 1/6841; C12Q 1/6853

(57) ABSTRACT

The present invention relates to functionalized single stranded oligonucleotides and in particular to a method for producing functionalized single stranded oligonucleotides comprising: (a) providing a circular DNA molecule comprising an oligonucleotide sequence bordered by cleavage domains; (b) performing a rolling circle amplification (RCA) reaction with the circular DNA molecule of (a) as a template and one or more functionalized nucleotides (dNTPs); and (c) enzymatically cleaving the product of the RCA reaction at the cleavage domains to release the single stranded functionalized oligonucleotides.

The present invention relates to a method for producing functionalized single stranded oligonucleotides. In particular, the invention provides a method that utilizes functionalized nucleotides in an enzyme-mediated rolling circle amplification (RCA) reaction to generate a plurality (e.g. a library) of functionalized oligonucleotides. A kit for use in the method and functionalized single stranded oligonucleotides obtained by the method are also provided. In particular, the invention provides a library comprising a plurality of functionalized single stranded oligonucleotides obtained by the method.

Single stranded nucleotides are useful in a wide range of applications due to their ability to hybridize, via Watson-Crick base pairing, to complementary sequences, e.g. intermolecularly. In addition, single stranded oligonucleotides can hybridize to themselves, (i.e. intramolecular complementarity) so as to form complex topological geometries and molecular assemblies, including secondary structures known as aptamers. These aptamers can bind with high affinity to biological targets via non-covalent interactions to effect a range of different functions. The utility of oligonucleotides could be enhanced by the addition of functionalities to the nucleotides, particularly on the nucleobases (i.e. the addition of reactive chemical groups), which do not interfere with the Watson-Crick base pairing. However, the synthesis of such functionalized single stranded oligonucleotides can be problematic, and this has inhibited the growth of the aforementioned applications.

At present, single stranded oligonucleotides are typically produced using solid-phase synthesis, wherein nucleotides are added in a stepwise manner to a growing chain bound to a solid support. However, this method can be severely limited in terms of the length and accuracy of the oligonucleotides produced. The error rate in the production of oligonucleotides by solid-phase synthesis is significantly higher than the error rate of polymerase enzymes observed in nature, and increases dramatically with the length of the oligonucleotide, such that purities of only 70% are common in commercial oligonucleotides of around 50 residues in length. Moreover, the availability of single stranded oligonucleotides containing a high density of functionalized nucleotides has been restricted due to limitations in terms of length, quality and costs of solid-phase synthesis. In particular, the number of functionalized nucleotides that can be incorporated into single stranded oligonucleotides produced by solid-phase synthesis is limited, e.g. due to cross-reactions caused by the functional groups.

Many of the uses of synthetic oligonucleotides require high purity levels, meaning additional steps of purification via methods such as high performance liquid chromatography (HPLC) or polyacrylamide gel electrophoresis (PAGE) are often required to isolate the desired products of solid-phase synthesis reactions. This makes the current methods both labour intensive and expensive. Moreover, even after these additional purification steps, practical issues attributable to remaining impurities can still be observed.

In view of these issues with solid-phase methods, there is a desire for alternative methods of producing functionalized oligonucleotides, particularly cost-efficient methods capable of producing longer oligonucleotides with greater accuracy.

Modified (e.g. functionalized) nucleotides have previously been used in enzymatic reactions to produce oligonucleotides, but only in the context of producing functionalized DNA via polymerase chain reactions (PCR) or primer extension (PE) reactions. These reactions necessitate the use of specific primers, which must then later be removed, and require not only that the modified nucleotides are incorporated by the polymerase, but also, in the case of PCR, that the functionalized products are recognized as templates. This can be problematic in the case of some modifications, and thus limits the utility of these methods. Moreover, the primers are synthesized by solid-phase methods and are not sequence verified, leading to the amplification of errors in the newly synthesized DNA. In addition, PCR-based methods result overwhelmingly in double stranded DNA products, and thus additional steps of elution and purification are necessary to obtain functionalized single stranded oligonucleotides.

The present inventors have previously described a method for enzymatic production of ‘monoclonal stoichiometric’ (MOSIC) single stranded DNA oligonucleotides from sequence-verified templates (Ducani et al., 2013, Nature Methods, 647-652). The inventors have surprisingly determined that the MOSIC method can be successfully adapted to produce single stranded functionalized oligonucleotides.

In a representative example, the method involves the design and preparation of a linear sequence comprising one or more oligonucleotide sequences, each bordered by hairpin sequences containing a restriction enzyme site. The linear sequence is then circularized into a double stranded rolling circle amplification (RCA) template, which is nicked and amplified by RCA in the presence of one or more functionalized nucleotides to produce a partially single stranded linear concatemer comprising functionalized nucleotides. The RCA product is then treated with a restriction enzyme which recognizes the restriction site in the aforementioned hairpin regions, and cleaves the concatemer to release a plurality of functionalized single stranded oligonucleotides, i.e. to produce a library of functionalized oligonucleotides.

As shown in the Examples, the inventors have advantageously determined that, contrary to expectations, functionalized nucleotides were efficiently incorporated by DNA polymerases with strand displacement activity, and did not prevent the formation of hairpin structures or interfere with their stability or effective cleavage. Moreover, the inventors found that functionalized nucleotides may be utilized in the MOSIC method whilst still maintaining the beneficial characteristics associated with the method. For instance, in contrast to the aforementioned PCR-based methods, the present method does not require primers and can produce functionalized single stranded oligonucleotides directly. This avoids the need for any additional steps to allow for primer annealing or to convert double stranded products into single stranded oligonucleotides. Moreover, the present method does not require the functionalized nucleotides to be recognized as a template for further amplification, which means that greater numbers of functionalized residues can be incorporated.

Accordingly, at its broadest, the invention can be seen to provide the use of a circular DNA molecule comprising an oligonucleotide sequence bordered by cleavage domains in the production of a single stranded functionalized oligonucleotide. More particularly, the invention may be viewed as the use of a circular DNA molecule comprising an oligonucleotide sequence bordered by cleavage domains and one or more functionalized nucleotides in the production of a single stranded functionalized oligonucleotide.

It will be evident that when the functionalized nucleotides are used in combination with the equivalent conventional nucleotide, the functionalized nucleotides may be incorporated randomly in the concatemer produced by the RCA reaction and thus cleavage of the concatemer results in a plurality (e.g. library) of single stranded functionalized oligonucleotides, i.e. where the functionalized nucleotides are incorporated in different positions in the oligonucleotides. It will be further evident that the diversity of the functionalized oligonucleotides produced may be increased by using a circular DNA molecule containing a plurality of oligonucleotide sequences, each bordered by cleavage domains. The oligonucleotide sequences may differ in their sequence and/or length. Additionally or alternatively, the diversity of the functionalized oligonucleotides may be increased by using a combination of functionalized nucleotides in the RCA reaction. Still further diversity may be introduced into the library of functionalized oligonucleotides by modifying the oligonucleotides after their synthesis, e.g. by conjugating molecules or components to the functionalized nucleotides in the oligonucleotides. Thus, it will be evident that the invention advantageously provides a new method for the enzymatic production of highly functionalized single stranded DNA oligonucleotides that may open new applications in DNA nanotechnology, nucleic acid imaging and bio-medicine. Thus, in one particular aspect, the present invention provides a method for producing single stranded functionalized oligonucleotides, said method comprising:

(a) providing a circular DNA molecule comprising an oligonucleotide sequence bordered by cleavage domains;

(b) performing a rolling circle amplification (RCA) reaction with the circular DNA molecule of (a) as a template and one or more functionalized nucleotides (dNTPs); and

(c) cleaving the product of the RCA reaction at the cleavage domains to release the single stranded functionalized oligonucleotides.

As discussed in more detail below, the step of providing a circular DNA molecule may be achieved by any suitable means and may depend on the structure of the circular DNA molecule. In this respect, the circular DNA molecule may be a single stranded DNA molecule or a double stranded DNA molecule.

In embodiments where the circular DNA molecule is a double stranded molecule, it will be evident that the circular DNA molecule must be processed to provide an RCA template. Thus, in some embodiments, the method comprises an additional step of cleaving a single strand of the circular DNA molecule to provide an RCA template, before the RCA reaction is performed.

The present invention advantageously may be used to produce functionalized oligonucleotides comprising any oligonucleotide sequence. Thus, any suitable sequence may be used as the oligonucleotide sequence in the circular DNA molecule of the invention. By a suitable sequence, it is meant that the oligonucleotide sequence domain should not interfere with (i.e. inhibit or distort) the production or cleavage of the RCA product. For instance, in some embodiments the oligonucleotide sequence may be designed to avoid the generation of secondary structures that may inhibit the progression of, or result in the displacement of, the polymerase performing the RCA reaction. Nevertheless, in some embodiments, the oligonucleotide sequence may be, or may encode, an aptamer.

Moreover, the oligonucleotide sequence may be designed such that it does not hybridize specifically to the cleavage domains in the RCA product. Alternatively viewed, the cleavage domains that border the oligonucleotide sequence may be designed such that they do not hybridize specifically to the oligonucleotide sequence(s) in the RCA product.

In embodiments where the circular DNA molecule comprises a plurality of oligonucleotide sequences, each sequence may be designed such that it does not hybridize specifically to the other oligonucleotide sequences in the RCA product. However, in some embodiments, it may be desirable to produce functionalized oligonucleotides containing regions of complementarity, e.g. to enable said oligonucleotides to interact, particularly following their release from the RCA product. Thus, in some embodiments, the oligonucleotide sequences may be designed to facilitate the interaction (e.g. hybridization) of functionalized oligonucleotides produced by the method insofar as such interactions do not interfere with the production or cleavage of the RCA product, i.e. the production of the functionalized oligonucleotides.

Thus, in some embodiments, the nucleic acid sequence of the oligonucleotide sequence of the circular DNA molecule has less than 80% sequence identity to the nucleic acid sequences in the cleavage domain and/or other oligonucleotide sequences in the circular DNA molecule. Preferably, the oligonucleotide sequence of the circular DNA molecule has less than 70%, 60%, 50% or less than 40% sequence identity to the nucleic acid sequences in the cleavage domain and/or other oligonucleotide sequences in the circular DNA molecule. Sequence identity may be determined by any appropriate method known in the art, e.g. the using BLAST alignment algorithm.

Thus, term “oligonucleotide sequence” is not particularly limiting and refers to the template sequence used to produce the functionalized single stranded oligonucleotide or oligonucleotides of the invention, or a complement thereof. In this respect, where the circular DNA molecule is single stranded, the sequence of the circular DNA molecule is the reverse complement of the repeated (tandem) sequence in the RCA product obtained in step (b). Where the circular DNA molecule is double stranded, it contains both the repeated sequence in the RCA product obtained in step (b) and its reverse complement. Accordingly, the step of processing the circular DNA to provide an RCA template may comprise a step of cleaving the strand of the circular DNA molecule containing the sequence that will be repeated in the RCA product.

The present invention may be used to produce functionalized single stranded oligonucleotides of any desired length. As shown in the Examples, the methods may be used to produce functionalized single stranded oligonucleotides that far exceed the length of oligonucleotides that can be accurately produced using solid-phase synthesis methods. Accordingly, an oligonucleotide sequence (and therefore functionalized oligonucleotide produced by the method) may be between about 6 up to about 10000 nucleotides in length. Thus, in some embodiments, the method may be viewed as the production of functionalized polynucleotides. In this respect, the boundary between the size of an “oligonucleotide” and “polynucleotide” is not well-defined in the art. For instance, a sequence of more than 400 nucleotides may be termed a polynucleotide. Accordingly, the terms oligonucleotide and polynucleotide are used interchangeably herein to refer to nucleotide sequences within the size range specified above. However, where the term “polynucleotide” is used, it will typically refer to sequences containing more than 400 nucleotides. Thus, in some embodiments, the method and use may be viewed as producing a plurality of single stranded functionalized DNA molecules.

In some embodiments, the oligonucleotide sequence may be from about 6 to about 750 nucleotides in length, including from about 6 to about 500 nucleotides in length, e.g., from about 6 to about 450 nucleotides in length, such as from about 8 to about 400 nucleotides in length, from about 8 to about 300 nucleotides in length, from about 8 to about 250 nucleotides in length, from about 10 to about 200 nucleotides in length, from about 10 to about 150 nucleotides in length, from about 12 to about 100 nucleotides in length, from about 12 to about 75 nucleotides in length, from about 14 to about 70 nucleotides in length, from about 14 to about 60 nucleotides in length, and so on. In some embodiments, the oligonucleotide sequence contain about 10-400, 11-390, 12-380, 13-370, 14-360 or 15-350 nucleotides.

As noted above, the invention is particularly effective in the production of longer oligonucleotides, e.g. comprising about 30 or more nucleotides, such as about 35, 40, 45, 50, 60, 70, 80, 90, 100 or more nucleotides. For instance, the oligonucleotides produced by the invention may contain about 30-1000, 40-900, 50-800, 60-700, 70-600, 80-500, 90-450 or 100-400 nucleotides. In some embodiments, the oligonucleotides (e.g. polynucleotides) produced by the invention may contain about 400-10000, 500-9000, 600-8000, 700-7000, 800-6000, 900-5000 or 1000-4000 nucleotides, e.g. comprising about 500, 1000, 1500, 2000, 2500, 3000, 3500 or more nucleotides.

In some embodiments, the circular DNA molecule comprises a plurality of oligonucleotide sequences, wherein each oligonucleotide sequence is bordered by cleavage domains. The oligonucleotide sequences may be the same or different from each other or a combination thereof. Thus, in some embodiments, the circular DNA molecule may comprise more than one copy of the same oligonucleotide sequence, e.g. 2, 3, 4, 5 etc. copies, as defined below. In some embodiments, the circular DNA molecule may comprise one copy each of a plurality of different oligonucleotide sequences, e.g. 2, 3, 4, 5 etc. different oligonucleotide sequences, as defined below. In some embodiments, the circular DNA molecule may comprise one or more copies of a plurality of different oligonucleotide sequences. As discussed further below, the present invention may be used to generate a plurality of functionalized oligonucleotides in controlled stoichiometry based on the number of copies of oligonucleotide sequences in the circular DNA molecule.

It will be evident that the plurality of oligonucleotide sequences in the circular DNA molecule may be present in any order. For instance, multiple copies of the same oligonucleotide sequence may be directly adjacent to each other in the circular DNA molecule (separated only by the cleavage domains that border the oligonucleotide sequences). Alternatively, multiple copies of the same oligonucleotide sequence may be interspersed with different oligonucleotide sequences. The circular DNA molecule may be designed (e.g. the order of the plurality of oligonucleotide sequences) to avoid or minimize interactions between the repeated sequences in the RCA product that may interfere with the production or cleavage of the RCA product as defined above.

As used herein, the term “plurality” means two or more, e.g. at least 2, 3, 4, 5, 6, 7, 8, 9, 10, 20 or 30 or more, such as 50, 100, 150, 200, 250 or more depending on the context of the invention. For instance, the circular DNA molecule may contain at least 2, 3, 4, 5, 6, 7, 8, 9, 10, 20 or 30 or more oligonucleotide sequences, such as 2-100, 3-90, 4-80, 5-70, 6-60, 7-50, 8-40, 9-30 or 10-20 oligonucleotide sequences. In some embodiments, the method of the invention may produce at least 2, 3, 4, 5, 6, 7, 8, 9, 10, 20 or 30 or more functionalized single stranded oligonucleotides, such as 50, 100, 150, 200, 250 or more, i.e. oligonucleotides with different sequences and/or structures. In this respect, different functionalized oligonucleotides may be produced from the same template oligonucleotide sequence because functionalized nucleotides may incorporated randomly in the RCA product, i.e. when the functionalized nucleotides are present in a relative amount of less than 100% as described above. Moreover, if the circular DNA molecule contains a plurality of different oligonucleotide sequences, each template will result in a plurality of different functionalized single stranded oligonucleotides. Furthermore, as a RCA reaction may utilize a plurality of circular DNA molecules, the reaction will result in a plurality of each functionalized single stranded oligonucleotide, e.g. 10³, 10⁴, 10⁵, 10⁶, 10⁷, 10⁸, 10⁹, 10¹⁰ or more copies of each functionalized single stranded oligonucleotide.

The term “different” refers to oligonucleotide sequences or functionalized single stranded oligonucleotides comprising one or more different nucleotides. Thus, different oligonucleotide sequences may differ by one or more, e.g. 2, 3, 4, 5, 6, 7, 8, 9, 10 or more, nucleotides, e.g. 20, 30, 40, 50, 60, 70, 80, 90 or more nucleotides. The differences may be in the length and/or sequence of the oligonucleotide sequence. Alternatively viewed, the different oligonucleotide sequences have less than 100% sequence identity each other, such as less than 99%, 98%, 97%, 96%, 95%, 90%, 85%, 80%, 70%, 60%, 50% to each other. Different functionalized single stranded oligonucleotides may comprise the same nucleotide sequences but differ in the position of functionalized nucleotides within the oligonucleotide or the type of functionalized nucleotide at a particular position (e.g. where a combination of functionalized nucleotides is used in the RCA reaction). Alternatively or additionally, different functionalized single stranded oligonucleotides may differ in the oligonucleotide sequence as defined above.

The present invention may therefore be used to produce a number of different single stranded functionalized oligonucleotides simultaneously. In particular, by altering the sequence of the circular DNA molecule, specifically, by controlling the number of copies of each different oligonucleotide sequence that are present, it is possible to control the stoichiometry of the single stranded functionalized oligonucleotides that are ultimately produced by the present method. However, as the functionalized single stranded oligonucleotides derived from each oligonucleotide sequence may differ (due to the random incorporation of functionalized nucleotides in the RCA product), the stoichiometry of the functionalized single stranded oligonucleotides may be controlled with respect to the number of oligonucleotides based on a particular oligonucleotide sequence. The circular DNA molecule may therefore comprise a plurality of copies of a plurality of different oligonucleotide sequences.

The term “cleavage domain” as used herein typically refers to a domain within the circular DNA molecule that results in a domain within the RCA product that can be cleaved specifically to release the functionalized single stranded oligonucleotides. Thus, the cleavage domain in the circular DNA molecule may be capable of cleavage directly or it may simply encode a cleavage domain that is only functional in the RCA product or functional under specific conditions, e.g. upon contact with a co-factor.

“Cleavage” includes any means of breaking a covalent bond. Thus, in the context of the invention, cleavage involves cleavage of a covalent bond in a nucleotide chain (i.e. strand cleavage or strand scission), for example by cleavage of a phosphodiester bond.

Thus, in some embodiments, a cleavage domain may comprise a sequence that is recognized by one or more enzymes capable of cleaving a nucleic acid molecule, i.e. capable of breaking the phosphodiester linkage between two or more nucleotides.

Accordingly, in some embodiments, the invention provides a method for producing single stranded functionalized oligonucleotides, said method comprising:

(a) providing a circular DNA molecule comprising an oligonucleotide sequence bordered by cleavage domains;

(b) performing a rolling circle amplification (RCA) reaction with the circular DNA molecule of (a) as a template and one or more functionalized nucleotides (dNTPs); and

(c) enzymatically cleaving the product of the RCA reaction at the cleavage domains to release the single stranded functionalized oligonucleotides.

Thus, in some embodiments, step (c) comprises contacting the product of the RCA reaction with a cleavage enzyme.

For instance, a cleavage domain may comprise a restriction endonuclease (restriction enzyme) recognition sequence. Restriction enzymes cut double-stranded or single stranded DNA at specific recognition nucleotide sequences known as restriction sites and suitable enzymes are well-known in the art. For example, it may be particularly advantageous to use rare-cutting restriction enzymes, i.e. enzymes with a long recognition site (at least 8 base pairs in length), to facilitate the design of the oligonucleotide sequence(s) in the circular DNA molecule, e.g. to avoid the inclusion of a cleavage recognition site within the oligonucleotide sequence(s).

In some embodiments, a cleavage domain may comprise a sequence that is recognized by a type II restriction endonuclease, more preferably a type IIs restriction endonuclease. While any suitable cleavage domain and cleavage enzyme may be used in the invention, in some embodiments the cleavage enzyme that recognizes a cleavage domain bordering the oligonucleotide sequences may be BseGI, BtsCI or an isoschizomer thereof, e.g. BstF5I or FokI. Other representative enzymes that may be used include BsrDI, BtsI, BtsIMutI, MlyI or isoschizomers thereof.

Thus, in some embodiments, the step of cleaving the product of the RCA reaction comprises contacting the RCA product with a cleavage enzyme under suitable conditions to selectively cleave the RCA product in the cleavage domains.

In some embodiments, a cleavage domain may be made functional (may be activated) in the RCA product by the addition of another component, i.e. the RCA product may be engineered to comprise a functional cleavage domain, e.g. a restriction endonuclease recognition sequence. For example, this may be achieved by hybridizing an oligonucleotide (termed herein a “restriction oligonucleotide”) to the cleavage domains of the RCA product to form a duplex. At least part of the formed duplex will comprise a restriction endonuclease recognition site, which can be cleaved resulting in the release of the functionalized oligonucleotides. This may be particularly advantageous in embodiments where the functionalized nucleotides incorporated into the RCA product, particularly the cleavage domains, may interfere with the activity (e.g. reduce the efficiency) of the cleavage enzyme.

Thus, in some embodiments, the step of cleaving the product of the RCA reaction may comprise contacting the RCA product with a restriction oligonucleotide and a cleavage enzyme. The restriction oligonucleotide and cleavage enzyme may be contacted with the RCA product simultaneously or sequentially.

In some embodiments, a cleavage domain may be cleaved by means other than a cleavage enzyme. For example, a cleavage domain may comprise a self-cleaving oligonucleotide sequence, such as a DNAzyme nuclease.

Thus, in some embodiments, the invention provides a method for producing single stranded functionalized oligonucleotides, said method comprising:

(a) providing a circular DNA molecule comprising an oligonucleotide sequence bordered by self-cleaving cleavage domains (e.g. cleavage domains comprising a DNAzyme nuclease);

(b) performing a rolling circle amplification (RCA) reaction with the circular DNA molecule of (a) as a template and one or more functionalized nucleotides (dNTPs); and

(c) cleaving the product of the RCA reaction at the cleavage domains to release the single stranded functionalized oligonucleotides,

wherein step (c) comprises activating the self-cleaving cleavage domains in the product of the RCA reaction.

Suitable self-cleaving sequences are known in the art. In embodiments that utilize a self-cleaving sequence, the cleavage domain may only be functional (active) in the RCA product. Alternatively, the self-cleaving sequence may be functional under specific conditions and thus the method may comprise a step of subjecting the RCA product to conditions that facilitate cleavage of the RCA product in the cleavage domains, i.e. conditions that activate the self-cleaving sequence, e.g. contacting the RCA product with a co-factor, such as metal ions, that are required for the self-cleaving activity. Alternatively viewed, the step of cleaving the product of the RCA reaction may comprise subjecting the RCA product to conditions that facilitate cleavage of the RCA product in the cleavage domains, i.e. conditions that activate the self-cleaving sequence, e.g. contacting the RCA product with a co-factor, such as metal ions, that are required for the self-cleaving activity.

In some embodiments, a cleavage domain comprises or consists of a sequence capable of forming a hairpin structure. A hairpin structure may also be known as a hairpin-loop or a stem-loop and these terms are used interchangeably herein. A hairpin is an intramolecular base-pairing pattern that can occur in a single-stranded DNA or RNA molecule. A hairpin occurs when two regions of the same strand, usually complementary in nucleotide sequence when read in opposite directions, base-pair (hybridize) to form a double stranded stem (a duplex) and an unpaired, i.e. single-stranded, loop. The resulting structure can be described as lollipop-shaped.

Thus, in some embodiments where the cleavage domain comprises or consists of a sequence capable of forming a hairpin structure, the cleavage domain comprises sequences that are self-complementary. As the RCA product extends, the hybridization of these self-complementary regions results in a hairpin structures, wherein the double-stranded portion of the hairpin structure comprises a sequence that is recognized by a cleavage enzyme. Thus, cleavage of the double-stranded portion of the hairpin structures in the RCA product results in the release of the functionalized single stranded oligonucleotides and hairpin structures (i.e. oligonucleotides that form the hairpin structures).

The term “hybridization” or “hybridizes” as used herein refers to the formation of a duplex between nucleotide sequences which are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary” under the invention. Usually two sequences are sufficiently complementary when at least about 90% (preferably at least about 95%) of the nucleotides share base pair organization over a defined length of the molecule.

Upon cleavage of the RCA product, the functionalized oligonucleotides are released. The functionalized oligonucleotides that are released may consist only of the oligonucleotide sequence (i.e. with no additional nucleotides), or they may comprise one or more additional nucleotides from the cleavage domains that border the oligonucleotide sequences at one or both ends. Thus in some embodiments, the functionalized oligonucleotides may comprise 1, 2, 3, 4, 5, 6, 7, 8, 9 or 10 or more nucleotides from the cleavage domains at one or both ends. Preferably, the sequence of the circular DNA molecule is designed such that when the RCA product is cleaved, the functionalized oligonucleotides that are released do not contain any additional nucleotides from the cleavage domains. Alternatively viewed, the cleavage domains that border the oligonucleotide sequence(s) may be arranged in the circular DNA molecule such that their cleavage in the RCA product results in the release of functionalized oligonucleotides that do not contain any additional nucleotides from the cleavage domains.

As cleavage enzymes may cleave a nucleic acid molecule at a position outside of the cleavage enzyme recognition sequence, there may be one or more nucleotides between a cleavage domain and an oligonucleotide sequence. Alternatively viewed, the cleavage domain may contain nucleotide sequences in addition to the cleavage enzyme recognition sequence to ensure that cleavage results in the release of the complete functionalized single stranded oligonucleotides, preferably without any additional nucleotides (e.g. nucleotides that form part of the cleavage domains).

Thus, the term “bordered” refers to cleavage domains that are directly or indirectly adjacent to the oligonucleotide sequence. Alternatively viewed, the cleavage domains are positioned at either end of the oligonucleotide sequence, i.e. the cleavage domains are upstream and downstream (at the 5′ and 3′ ends) of the oligonucleotide sequence. In some embodiments, the cleavage site of the cleavage domains (e.g. the site at which a cleavage enzyme cleaves a cleavage domain) is directly adjacent to the end of the oligonucleotide sequence it borders. In some embodiments, the oligonucleotide sequence and cleavage domain sequence may overlap, i.e. the end of the oligonucleotide sequence may form part of the cleavage domain, e.g. when the cleavage site is an internal site within the cleavage domain, i.e. the cleavage domains may form the ends or part of the ends of the oligonucleotide sequence. Thus, in some embodiments, there may be one or more, e.g. 1, 2, 3, 4, 5, 6, 7, 8, 9 or 10 or more, nucleotides between the cleavage domain and the oligonucleotide sequence (i.e. between the ends of the sequences). In some embodiments, the cleavage domain and the oligonucleotide sequence may overlap one or more, e.g. 1, 2, 3, 4, 5, 6, 7, 8, 9 or 10 or more, nucleotides.

The size of the cleavage domains in the circular DNA molecule is not particularly limited and will depend on the type of cleavage domain, as described above. The cleavage domain may be designed or selected such that the length of the cleavage domain is different from the length of the oligonucleotide sequences, to allow for the functionalized single stranded oligonucleotide sequences to be easily purified once the RCA product has been cleaved at the cleavage domain. Thus, in some embodiments, the cleavage domain(s) may be selected to be shorter than the oligonucleotide sequence in the circular DNA molecule. If the circular DNA molecule contains a plurality of oligonucleotide sequences of different lengths, the cleavage domain(s) may be selected to be shorter than the shortest oligonucleotide sequence in the circular DNA molecule. In other embodiments, the cleavage domain(s) may be selected to be longer than the oligonucleotide sequence in the circular DNA molecule. If the circular DNA molecule contains a plurality of oligonucleotide sequences of different lengths, the cleavage domain(s) may be selected to be longer than the longest oligonucleotide sequence in the circular DNA molecule. In some embodiments, the length of the cleavage domain(s) and the oligonucleotide sequences differ by at least 2 nucleotides, such as at least 3, 4, 5, 6, 7, 8, 9 or 10 nucleotides.

If the desired oligonucleotide sequences and the cleavage domains are of approximately the same length, the cleavage domains may be extended so that they can be separated from the final single stranded functionalized oligonucleotides by size. For example, additional regions of self-complementarity may be added to the ends of the cleavage domains so as to extend the length of the stem region of the hairpin structure. Alternatively or additionally, nucleotides may be added to the loop region of the hairpin structure.

In some embodiments, cleavage domains may be about 4-50 nucleotides in length, such as about 5-45, 6-40, 7-35 or 8-30 nucleotides in length. In some embodiments they may range from about 10 to 25 nucleotides in length, including from about 12 to 22 or from about 14 to 20. However, it will be evident that any suitable length of cleavage domain may be used in the invention as long as it meets the functional requirements described above.

The cleavage domains that border the oligonucleotide sequences may be the same or different from each other. Advantageously, the cleavage domains that border the oligonucleotide sequence(s) are the same such that a single cleavage step is sufficient to release all of the functionalized single stranded oligonucleotides. For instance, in some embodiments, the step of cleaving the RCA product comprises contacting the RCA product with a single cleavage enzyme under conditions suitable to cleave the cleavage domains in the RCA product.

Suitable conditions to cleave the cleavage domains in the RCA product will be dependent on the means used to achieve cleavage. For instance, where cleavage is achieved using a cleavage enzyme, such as a restriction endonuclease, conditions will differ depending on the enzyme selected and suitable conditions are well-known in the art, e.g. the cleavage step may follow the manufacturer's instructions. Similarly, where cleavage is achieved using a self-cleaving sequence, such as a DNAzyme, conditions appropriate for the particular sequence may be used. An example of a range suitable conditions that may be used in the cleavage step is set out below.

For instance, a cleavage enzyme, e.g. a restriction endonuclease, may specifically bind to its cleavage recognition site and selectively (e.g. specifically) cleave the nucleic acid in a variety of buffers, such as phosphate buffered saline (PBS), 4-(2-hydroxyethyl)-1-piperazineethanesulfonic acid (HEPES), HEPES buffered saline (HBS), and Tris buffered saline (TBS), both with and without EDTA. Cleavage may occur in a pH range of about 3.0-10.0, e.g. 4.0-9.0, 5.0-8.0, over a wide range of temperatures, e.g. 0-70° C. The skilled person would readily be able to determine other suitable conditions.

The method of the invention comprises a step of performing rolling circle amplification using the circular DNA molecule as a template. Rolling-circle amplification (RCA) is well-known in the art, being described in Dean et al., 2001 (Rapid Amplification of Plasmid and Phage DNA Using Phi29 DNA Polymerase and Multiply-Primed Rolling Circle Amplification, Genome Research, 11, pp. 1095-1099), the disclosures of which are herein incorporated by reference. In brief, RCA relates to the synthesis of nucleic acid molecules using a circular single stranded nucleic acid molecule, e.g. a circle or circular oligonucleotide, as rolling circle template (a RCA template) and a strand-displacing polymerase to extend a primer which is hybridized to the template. The addition of a polymerase and nucleotides starts the synthesis reaction, i.e. polymerization. As the rolling circle template is endless, the resultant product is a long single stranded nucleic acid molecule composed of tandem repeats that are complementary to the rolling circle template.

A typical RCA reaction mixture includes a circular DNA molecule and one or more primers that are employed in the primer extension reaction, e.g. RCA may be templated by a single primer to generate a single concatemeric product or multiple primers, each annealing to a different region of the circular oligonucleotide to produce multiple concatemeric products per circle. The oligonucleotide primers with which the circular nucleic acid may be contacted will be of sufficient length to provide for hybridization to the circular DNA molecule under annealing conditions. In some embodiments, the primer may be provided by cleaving (e.g. nicking) a single strand of a double stranded circular DNA molecule provided in step (a).

In addition to the above components, the reaction mixture used in the invention typically includes a polymerase (defined further below, e.g. phi29 DNA polymerase), one or more functionalized nucleotides, one or more conventional nucleotides and other components required for a DNA polymerase reaction as described below. The desired polymerase activity may be provided by one or more distinct polymerase enzymes.

The RCA reaction mixture may further include an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulphate, and the like may be employed. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, but will preferably range from about 3 to 6 mM, and will ideally be at about 5 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5. Other agents which may be present in the buffer medium include chelating agents, such as EDTA, EGTA and the like.

In embodiments in which a single strand of the double stranded circular DNA molecule is cleaved (e.g. nicked) to provide the RCA template (and the primer for the RCA reaction), it may be useful to include a single stranded binding protein in the RCA reaction mixture. For example, E. coli single stranded DNA binding protein has been used to increase the yield and specificity of primer extension reactions and PCR reactions. (U.S. Pat. Nos. 5,449,603 and 5,534,407). The gene 32 protein (single stranded DNA binding protein) of phage T4 apparently improves the ability to amplify larger DNA fragments (Schwartz, et al., Nucl. Acids Res. 18: 1079 (1990)), it enhances DNA polymerase fidelity (Huang, DNA Cell. Biol. 15: 589-594 (1996)) and, most importantly, it prevents DNA polymerase from switching templates, e.g. to the already produced single stranded DNA, and synthesizing double stranded DNA (Ducani et al. Nucl. Acids Res. 42: 10596 (2014)). When employed, such a protein will be used to achieve a concentration in the reaction mixture that ranges from about 0.01 ng/μL to about 1 μg/μL; such as from about 0.1 ng/μL to about 100 ng/μL; including from about 1 ng/μL to about 10 ng/μL.

The RCA reaction ultimately produces a polynucleotide product comprising adjacent (tandem) repeats of the complementary sequence of the circular DNA molecule. This product may be known as a concatemer, an RCA product or “RCP”. The RCA product therefore comprises a linear sequence made up of oligonucleotide sequences (or more particularly the reverse complement of the oligonucleotide sequences of the circular DNA molecule template) comprising functionalized nucleotides, bordered by cleavage domains.

As noted above, where the circular DNA molecule is single stranded, the RCA reaction mixture may comprise one or more oligonucleotide primers, which initiate the RCA polymerization reaction. The primers will be of sufficient length to provide for hybridization to the circular DNA molecule under annealing conditions. The primers will generally be at least 10 nucleotides in length, usually at least 15 nucleotides in length and more usually at least 16 nucleotides in length and may be as long as 30 nucleotides in length or longer, where the length of the primers will generally range from 18 to 50 nucleotides in length, usually from about 20 to 35 nucleotides in length.

The primers may anneal to any region within the circular DNA molecule. In some embodiments, the circular DNA molecule may comprise a specific domain (a RCA primer binding site) to which the primer may hybridize. In a representative embodiment, the circular DNA molecule may comprise a sequence between the cleavage domains that border the oligonucleotide sequence, which is not the oligonucleotide sequence and which may function as the RCA primer binding site. The RCA primer binding site may be designed to be of a different length to the oligonucleotide sequence, akin to the cleavage domains discussed above, to ensure that it can be readily separated from the functionalized single stranded oligonucleotides upon cleavage of the RCA product. It will be evident that in a circular DNA molecule comprising a plurality of oligonucleotide sequences bordered by cleavage domains, the RCA primer binding site may be between any two cleavage domains.

The term “annealing conditions” refers to the conditions under which two nucleic acid molecules comprising complementary nucleotide sequences will specifically hybridize to each other. Various parameters affect hybridization including temperature, salt concentration, nucleic acid concentration, composition and length, and buffer composition. The skilled person readily can determine suitable annealing conditions for a particular primer/template combination for an RCA reaction as a matter of routine.

As noted above, in some embodiments, the circular DNA molecule is double stranded and the method comprises an additional step of cleaving a single strand of the circular DNA molecule to provide an RCA template, before the RCA reaction is performed. It will be evident that the step of cleaving a single strand of the circular DNA molecule may replace the step of providing a primer to initiate the RCA reaction, i.e. the cleaved strand functions as the RCA primer. Thus, alternatively viewed, the method comprises an additional step of cleaving a single strand of the circular DNA molecule to provide an RCA template and primer, before the RCA reaction is performed, i.e. the introduction of a single strand break in the circular DNA molecule creates a 3′ end which can act as a primer for the RCA reaction. However, in some embodiments, it may be advantageous to provide one or more RCA primers in the reaction mixture in addition to cleaving one strand of the double stranded circular DNA molecule, e.g. to increase the number of RCA products obtained per circle.

In some embodiments, the step of cleaving a single strand of the circular DNA molecule to provide an RCA template comprises cleaving a single strand of the circular DNA molecule with a cleavage enzyme. This cleavage of a single strand of the circular DNA molecule results in a single strand break (a nick).

In some embodiments, it may be advantageous to cleave a single strand of the circular DNA molecule multiple times in close proximity, in order to facilitate the binding of the DNA polymerase to the 3′ end generated by the cleavage. Accordingly, a single strand of the circular DNA molecule may be cleaved two or more times, i.e. two or more nicks may be generated, in close proximity to each other. Preferably, the nicks are generated within 20 nucleotides, more preferably within 10 nucleotides, more preferably within 5 nucleotides of each other, e.g. within 1, 2, 3, 4 or 5 nucleotides of each other.

In some embodiments, the cleavage enzyme used to cleave one strand of the double stranded circular DNA molecule is a nickase. Nickases are endonucleases which cleave only a single strand of a DNA duplex. Some nickases introduce single-stranded nicks only at particular sites on a DNA molecule, by binding to and recognizing a particular nucleotide recognition sequence. A number of naturally-occurring nickases have been discovered, of which at present the sequence recognition properties have been determined for at least four. Nickases are described in U.S. Pat. No. 6,867,028, which is herein incorporated by reference in its entirety and any suitable nickase may be used in the methods of the invention. In some embodiments, the cleavage enzyme (nickase) may be Nb.BsrDI, Nt.BspQI or a combination thereof.

In some embodiments that utilize a nickase enzyme, the nickase enzyme may be removed from the assay or inactivated following cleavage of the circular DNA molecule to prevent unwanted cleavage of RCA products.

The cleavage enzyme that cleaves a single strand of the circular DNA molecule (e.g. nickase) may cleave at any site within the circular DNA molecule. In some embodiments, the circular DNA molecule may comprise a specific domain (a single strand cleavage site or domain, e.g. a nickase site) at which the cleavage enzyme may act. In a representative embodiment, the circular DNA molecule may comprise a sequence between the cleavage domains, which is not the oligonucleotide sequence and which may function as the single strand cleavage site or domain. The fact that the sequence recognized by the cleavage enzyme (the single strand cleavage site or domain) is not in the oligonucleotide sequence ensures that the oligonucleotide produced from the 5′ end of the RCA product is not truncated. The single strand cleavage site or domain may be designed to be of a different length to the oligonucleotide sequence, akin to the cleavage domains and RCA primer binding site discussed above, to ensure that it can be readily separated from the functionalized single stranded oligonucleotides upon cleavage of the RCA product. Thus, in some embodiments, the RCA primer binding site also functions as the single strand cleavage site or domain or vice versa.

Any DNA polymerase with at least some strand displacement activity may be used in the RCA reaction of the invention. Strand displacement activity ensures that once the polymerase has extended around the circular DNA molecule, it can displace the primer sequence and the elongating product and continue to “roll” around the template. In embodiments where the nicked strand of the circular DNA molecule provides the primer for RCA extension, the strand displacement activity ensures that the polymerase can displace the nicked strand. Suitable DNA polymerase enzymes with at least some strand displacement activity include phi29 DNA polymerase, E. coli DNA polymerase I, Bsu DNA polymerase (large fragment), Bst DNA polymerase (large fragment) and Klenow fragment. As used herein, the term “DNA polymerase” includes not only naturally occurring enzymes but also all such modified derivatives, including also derivatives of naturally occurring DNA polymerase enzymes. For instance, in some embodiments, the DNA polymerase may have been modified to remove 5′-3′ exonuclease activity.

Particularly preferred DNA polymerase enzymes for use in the invention include phi29 DNA polymerase, Bst DNA polymerase and derivatives, e.g. sequence-modified derivatives, or mutants thereof.

Sequence-modified derivatives or mutants of DNA polymerase enzymes include mutants that retain at least some of the functional activity, e.g. DNA polymerase activity and at least some strand displacement activity, of the wild-type sequence. Mutations may affect the activity profile of the enzymes, e.g. enhance or reduce the rate of polymerization, under different reaction conditions, e.g. temperature, template concentration, primer concentration etc. Mutations or sequence-modifications may also affect the exonuclease activity and/or thermostability of the enzyme.

As noted above, the RCA reaction of the present method is typically conducted using a mixture of functionalized and conventional nucleotides.

The term “conventional nucleotides” as used herein refers to deoxynucleotides comprising one of the four bases found in DNA; adenine, guanine, cytosine and thymine. The term “conventional nucleotides” thus encompasses, for example, dATP, dGTP, dCTP and dTTP. Whilst uracil is not typically found in DNA naturally, dUTP readily may be used instead of, or in addition to, dTTP. Thus, in the context of the present invention, dUTP may be viewed as a “conventional” nucleotide. Usually the reaction mixture will include at least three of the four different types of dNTPs corresponding to the four naturally occurring bases present, i.e. dATP, dTTP, dCTP and dGTP. However, as noted above, dUTP may be used instead of, or in addition to, dTTP. Moreover, in some embodiments, the reaction mixture may include four or five dNTPs, i.e. dATP, dCTP, dGTP, dTTP and/or dUTP. In the subject methods, each dNTP will typically be present in an amount ranging from about 10 to 5000 μM, usually from about 20 to 1000 μM. Each dNTP may be present in the different amounts or an equal amount of each dNTP may be used.

The terms “functionalized nucleotides” or “functionalized dNTPs” refer to nucleotides that comprise a modification relative to unmodified conventional nucleotides, wherein said modification provides said functionalized nucleotides and/or oligonucleotides comprising at least one functionalized nucleotide with additional or alternative properties or characteristics, i.e. relative to the corresponding conventional nucleotide. For instance, the modification may render the nucleotide detectable, e.g. by the incorporation of a label, or capable of interacting and/or reacting with another component, i.e. a component with which the corresponding conventional nucleotide does not interact or react. In some embodiments, the modification may render the oligonucleotide containing the nucleotide resistant to degradation, e.g. chemical and/or enzymatic degradation (e.g. nuclease degradation), or may alter the metabolism of the nucleotide.

The terms “functionalized single stranded oligonucleotide”, “single stranded functionalized oligonucleotide” and “functionalized oligonucleotide” are used interchangeably herein and refer to a single stranded oligonucleotide containing at least one functionalized nucleotide. Thus, a functionalized oligonucleotide has additional or alternative properties or characteristics relative to the corresponding oligonucleotide containing only conventional nucleotides. For instance, the incorporation of one or more functionalized nucleotides may render the oligonucleotide detectable, e.g. by the incorporation of a label, or capable of interacting and/or reacting with another component, i.e. a component with which the corresponding oligonucleotide containing only conventional nucleotides does not interact or react. In some embodiments, the modification may render the oligonucleotide resistant to degradation, e.g. chemical and/or enzymatic degradation (e.g. nuclease degradation), or may alter the metabolism of the oligonucleotide. In some embodiments, the modification may improve the stability of the oligonucleotide, e.g. improve the stability of duplexes formed by the oligonucleotide, such as the thermostability of the duplexes (e.g. melting temperature) In some embodiments, the incorporation of one or more functionalized nucleotides may render the oligonucleotide capable of forming secondary or tertiary structures that are not formed by a corresponding oligonucleotide containing only conventional nucleotides.

Accordingly, it will be understood that in the context of a functionalized single stranded oligonucleotide, the term “single stranded” refers to an oligonucleotide which is single stranded under denaturing conditions, e.g. following the application of heat or suitable chemical denaturing agents, i.e. an oligonucleotide with only one continuous backbone (one strand). As noted above, this does not preclude a functionalized single stranded oligonucleotide from forming secondary or tertiary structures. For example, the functionalized single stranded oligonucleotide may comprise regions of self-complementarity, and thus may be capable of forming a hairpin or stem-loop structure, mediated by one region of the functionalized single stranded oligonucleotide hybridizing to a complementary region elsewhere in the same oligonucleotide.

Functionalisation of a conventional nucleotide may be achieved by alteration of, or modification to, the structure of any part of the nucleotide. Thus, a functionalized nucleotide may contain a modification, e.g. a chemical modification, on the nucleobase, sugar or group involved in the internucleotide linkage. In some preferred embodiments, the functionalized nucleotide contains a modification, e.g. a chemical modification, on the nucleobase. Modifications at various positions are described in more detail below and it is contemplated that any of the specific positions described below may be modified with any of functional groups described below.

For instance, the substitution of the C5 position in pyrimidines (i.e. cytosine, thymine and uracil) with a group that is small, rigid and hydrophobic may improve base stacking interactions of, and/or stabilize duplexes formed by, oligonucleotides comprising functionalized nucleotides containing the substituted pyrimidines. A small, rigid and hydrophobic group may be an alkynyl group, e.g. methyl, ethynyl, propynyl, or a halogen group, e.g. fluoro, chloro or bromo. Thus, in some embodiments, the functionalized nucleotide comprises a pyrimidine with a substitution at the C5 position, e.g. an alkynyl group or halogen as defined above.

In some embodiments, a modification that may render the nucleotide detectable may involve the incorporation of a label into the nucleotide. Any label may find utility in the invention and may be a directly or indirectly signal giving molecule. For instance, directly signal giving labels may be fluorescent molecules, i.e. the functionalized nucleotides may be fluorescently labeled nucleotides. Indirectly signal giving labels may be, for example, biotin molecules, i.e. the labeled nucleotides may be biotin labeled nucleotides, which require additional steps to provide a signal, e.g. the addition of streptavidin conjugated to an enzyme which may act on a chemical substrate to provide a detectable signal, e.g. a visibly detectable colour change. In some embodiments, the label is incorporated in (conjugated to) the nucleobase.

Thus, in some embodiments, the functionalized nucleotide comprises a biotin group conjugated to a nucleobase. In some embodiments, the biotin group may be conjugated to the nucleobase indirectly, e.g. via a linker or linking domain and a suitable linker may be readily selected from those well-known in the art. For instance, the linker may be selected to facilitate the interaction between biotin and streptavidin, i.e. to minimize or prevent steric hindrance. In some embodiments, the biotin group is conjugated to a pyrimidine at the C5 position. In a representative embodiment, the biotin containing functionalized nucleotide may be Biotin-16-Aminoallyl-2′-dUTP, Biotin-16-Aminoallyl-2′-dTTP or Biotin-16-Aminoallyl-2′-dCTP.

In some embodiments, the nucleotide may be labeled with a sterol group, i.e. the nucleotide may comprise a sterol group. In some embodiments, the nucleotide may be labeled with or may comprise a cholesterol group.

A directly detectable label is one that can be directly detected without the use of additional reagents, while an indirectly detectable label is one that is detectable by employing one or more additional reagents, e.g. where the label is a member of a signal producing system made up of two or more components. In many embodiments, the label is a directly detectable label, where directly detectable labels of interest include, but are not limited to: fluorescent labels, coloured labels, radioisotopic labels, chemiluminescent labels, and the like. Any spectrophotometrically or optically-detectable label may be used. In other embodiments the label may provide a signal indirectly, i.e. it may require the addition of further components to generate signal. For instance, the label may be capable of binding a molecule that is conjugated to a signal giving molecule.

In some embodiments, the functionalized nucleotide is a fluorescently labeled nucleotide. Whilst fluorescent labels require excitation to provide a detectable signal, as the source of excitation is derived from the instrument/apparatus used to detect the signal, fluorescent labels may be viewed as directly signal giving labels.

Fluorescent molecules that may be used to label nucleotides are well known in the art. Fluorophores have been identified with excitation and emission spectra ranging from UV to near IR wavelengths. Thus, the fluorophore may have an excitation and/or emission wavelength in the UV, visible or IR spectral range.

The fluorophore may be a protein, peptide, small organic compound, synthetic oligomer or synthetic polymer. In some embodiments, the fluorophore is a small organic compound, e.g. an organic compound with a molecular weight of 5000 Da or less. Thus, in some embodiments, the fluorophore has a molecular weight of 4000 Da or less, such as 3500 Da, 3000 Da, 2500 Da, 2250 Da, 2000 Da, 1900 Da, 1800 Da, 1700 Da, 1600 Da, 1500 Da or less.

Thus, the fluorophore may be a xanthene derivative (e.g. fluorescein, rhodamine, Oregon green, eosin, Texas red), a cyanine derivative (e.g. cyanine, indocarbocyanine, oxacarbocyanine, thiacarbocyanine, merocyanine), a squaraine derivative (e.g. ring-substituted squaraine, Seta, SeTau), a naphthalene derivative (e.g. dansyl or prodan derivative), a coumarin derivative, an oxadiazole derivative (such as pyridyloxazole, nitrobenzoxadiazole and benzoxadiazole), an anthracene derivative (such as an anthraquinone, including DRAQ5, DRAQ7 and CyTRAK Orange), a pyrene derivative (e.g. cascade blue), an oxazine derivative (e.g. Nile red, Nile blue, cresyl violet, oxazine 170), an acridine derivative (such as proflavin, acridine orange, acridine yellow), an arylmethine derivative (e.g. auramine, crystal violet, malachite green) or a tetrapyrrole derivative (such as porphyrin, phthalocyanine, bilirubin).

Specific examples of fluorophores or fluorophore series that may find utility in the present invention include Alexa Fluors (such as Alexa Fluor 488, Alexa Fluor 647 etc.), Atto, Cyanine (Cy), indocyanine, sulfocyanine, DyLight, Abberior STAR, Chromeo, Oregon Green, Fluorescein, Texas red, Rhodamine, Silicon-rhodamine (SiR), Squaraine, FluoProbes, Tetrapyrrole, Bodipy, HiLyte, Quasar, CAL fluor, Coumarin, Seta, CF, Tracy, IRDye, CruzFluor, Tide Fluor, Oyster, iFluor, Chromis and Brilliant Violet and fluorescent derivatives or analogues thereof.

In some embodiments, the functionalized nucleotide contains a cyanine fluorescent label, such as Cy3. In some particular embodiments, the functionalized nucleotide is dATP labeled with Cy3, e.g. 7-Propargylamino-7-deaza-ATP-Cy3.

In some embodiments, the functionalized nucleotide contains an atto fluorescent label, such as atto-488. In some particular embodiments, the functionalized nucleotide is dATP labeled with atto-488, e.g. 7-Propargylamino-7-deaza-ATP-Atto-488.

In some embodiments, a modification that may render the nucleotide reactive involves the incorporation of a reactive group, e.g. a group capable of forming a covalent bond with another chemical group, e.g. a chemical group on a molecule or component to be conjugated to the functionalized oligonucleotide, such as a label as defined herein. Potential reactive groups include nucleophilic functional groups (alkynes, alkenyls, amines, alcohols, thiols, hydrazides, azides), electrophilic functional groups (aldehydes, esters, vinyl ketones, epoxides, isocyanates, maleimides), functional groups capable of cycloaddition reactions, forming disulfide bonds, or binding to metals. Specific examples include ethyne (acetylene), propyne, 1-butyne, 2-butyneazide, vinyl (ethenyl), propenyl, 1-butenyl, primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, azides and maleimides.

In some embodiments, a modification that may render the nucleotide reactive involves the incorporation of a reactive group that is capable of reacting with another chemical group, e.g. a chemical group on a molecule or component to be conjugated to the functionalized oligonucleotide, via click chemistry. As used herein, the term “click chemistry,” generally refers to reactions that are modular, wide in scope, give high yields, generate only inoffensive by-products, such as those that can be removed by nonchromatographic methods, and are stereospecific (but not necessarily enantioselective). See, e.g., Angew. Chem. Int. Ed., 2001, 40(11):2004-2021, which is entirely incorporated herein by reference. In some cases, click chemistry can describe pairs of functional groups that can selectively react with each other in mild, aqueous conditions. Accordingly, click chemistry groups are suitable for the conjugation of additional functional groups to the oligonucleotides of the present method. Common click chemistry reactions include azide-alkyne cycloadditions, alkyne-nitrone cycloadditions, alkene-tetrazine reactions and alkene-tetrazole reactions. Accordingly, the functionalized nucleotide may comprise an azide group, an alkyne group, an alkene group, a nitrone group, a tetrazine group or a tetrazole group.

A specific example of click chemistry reaction can be the Huisgen 1,3-dipolar cycloaddition of an azide and an alkyne, i.e., Copper-catalyzed reaction of an azide with an alkyne to form a 5-membered heteroatom ring called 1,2,3-triazole. The reaction can also be known as a Cu(I)-Catalyzed Azide-Alkyne Cycloaddition (CuAAC), a Cu(I) click chemistry or a Cu+ click chemistry. Catalyst for the click chemistry can be Cu(I) salts, or Cu(I) salts made in situ by reducing Cu(II) reagent to Cu(I) reagent with a reducing reagent (Pharm Res. 2008, 25(10): 2216-2230). Known Cu(II) reagents for the click chemistry can include, but are not limited to, Cu(II) (TBTA) complex and Cu(II) (THPTA) complex. TBTA, which is tris-[(1-benzyl-1H-1,2,3-triazol-4-yl)methyl]amine, also known as tris-(benzyltriazolylmethyl)amine, can be a stabilizing ligand for Cu(I) salts. THPTA, which is tris-(hydroxypropyltriazolylmethyl)amine, can be another example of stabilizing agent for Cu(I). Other conditions can also be accomplished to construct the 1,2,3-triazole ring from an azide and an alkyne (e.g. a cycloalkyne, such as cyclooctyne or cyclononyne) using copper-free click chemistry, such as by the Strain-promoted Azide-Alkyne Click chemistry reaction (SPAAC, see, e.g., Chem. Commun., 2011, 47:6257-6259 and Nature, 2015, 519(7544):486-90), each of which is entirely incorporated herein by reference.

In some embodiments, the functionalized nucleotide contains a reactive group in the in the sugar group, e.g. a modification at position 2 in the deoxyribose sugar, such as substituting the hydrogen with a fluoro, chloro, bromo or azide group. In some embodiments, the functionalized nucleotide is a 2′-Azido-dNTP, e.g. 2′-Azido-dATP.

In some embodiments, the functionalized nucleotide contains a reactive group in the nucleobase, particularly selected from an alkyne, alkenyl, thio or halogen group. As noted above, the functionalized nucleotide may comprise a pyrimidine comprising a substitution at the C5 position. In some embodiments, the alkyne group is ethyne, e.g. the functionalized nucleotide is an ethynyl-dNTP, such as 5′-Ethynyl-dUTP. Alternatively, the alkyne group may be a propynyl group, e.g. the functionalized nucleotide is a propynyl dNTP, such as 5′-Propynyl-dUTP. In some embodiments, the alkenyl group is vinyl (ethenyl), e.g. the functionalized nucleotide is a vinyl-dNTP, such as 5′-vinyl-dUTP. In some embodiments, the functionalized nucleotide is a thio-dNTP, such as 4′-thio-dTTP. In some embodiments, the halogen group is bromine, e.g. the functionalized nucleotide is a bromo-dNTP, such as 5′-Bromo-dUTP. The incorporation of halogen groups into functionalized oligonucleotides can be used to promote nucleophilic aromatic substitutions or UV mediated crosslinking, e.g. with proteins. Thus, in some embodiments, functionalized oligonucleotides produced by the present method may be further modified to comprise aromatic groups or may by conjugated to other molecules, e.g. proteins or peptides, via UV mediated crosslinking.

In some embodiments, a functionalized nucleotide may contain a group capable of interacting with another component, e.g. interacting via a non-covalent bond. For instance, the nucleotide may be modified to incorporate one part or component of a cognate binding pair, e.g. an affinity binding partner, e.g. biotin or a hapten, capable of binding to its binding partner, i.e. a cognate binding partner, e.g. streptavidin or an antibody. Such functionalized nucleotides may, for example, find utility in the production of functionalized oligonucleotides that may be immobilized on a solid support.

In some embodiments, the functionalized nucleotide contains a modification that renders the oligonucleotide containing the nucleotide resistant to degradation, e.g. chemical and/or enzymatic degradation (e.g. nuclease degradation). In some embodiments, the nucleotide contains a modification in the sugar group, e.g. a modification at position 2 in the deoxyribose sugar, such as substituting the hydrogen with a fluoro, chloro, O-methyl or O-ethyl group. Thus, in some embodiments, the functionalized nucleotide is a 2′-fluoro-dNTP, e.g. 2-fluoro-UTP. In some embodiments the functionalized nucleotide comprises an O-Me group, e.g. 2′-O-Methyl-ATP. In some embodiments, the nucleotide contains a modification in the phosphate group that forms the internucleotide linkage, such as substituting an oxygen with a sulfur, e.g. the nucleotide contains a phosphorothionate group. Thus, in some embodiments, the functionalized nucleotide is nucleotide thiotriphosphate, e.g. 2-deoxythymidine-5′-O-(1-thiotriphosphate), 2-deoxycytidine-5′-O-(1-thiotriphosphate), 2-deoxyuridine-5′-O-(1-thiotriphosphate), 2-deoxyadenosine-5′-O-(1-thiotriphosphate) or 2-deoxyguanosine-5′-O-(1-thiotriphosphate). In some embodiments, the functionalized nucleotides contains a modification in the nucleobase, such as an amino, methyl, ethyl or propynyl modification, e.g. 2-amino-dATP, 5-methyl-dCTP, C-5 propynyl-dCTP or C-5 propynyl-dUTP.

In some embodiments, the functionalized nucleotide contains a modification that affects the thermostability of the oligonucleotide. In some embodiments, the nucleotide comprises a locked ribose sugar, i.e. comprises an additional covalent bond between the 2′ oxygen and the 4′ carbon of the pentose ring. In some embodiments, the nucleotide is an LNA (locked nucleic acid) nucleotide, i.e. LNA-NTP such as LNA-ATP. The locked ribose conformation enhances base stacking and thus increases the melting temperature of oligonucleotides comprising LNA nucleotides.

Thus, in some embodiments, the functionalized nucleotides that may be used in the invention include: nucleotides comprising an (internalized) alkyne or azide group, fluorescently labeled nucleotides, nucleotides comprising a sterol group, nucleotides comprising a polyether group, nucleotides comprising a metal complex, nucleotides comprising a vinyl group, nucleotides comprising a thiol group, thionated nucleotides, nucleotides modified to have increased nuclease resistance, nucleotides comprising a chemical group capable of participating in click chemistry, nucleotides that affect (e.g. increase) the thermostability of the oligonucleotide (e.g. LNA-nucleotides) or a combination thereof. The incorporation of these functionalized nucleotides into the single stranded oligonucleotides of the present invention can provide a range of useful functions. For example, single stranded oligonucleotides comprising fluorophores can be used as sequence specific fluorescent probes. The inclusion of thiolated nucleotides in a single stranded oligonucleotide allows the oligonucleotide to be labeled with thiol-reactive molecules, and to be used as a probe for molecular detection of such thiol-reactive molecules.

In some embodiments, the functionalized nucleotides that may be used in the invention do not include nucleotides comprising a digoxigenin group. Alternatively put, in some embodiments, the functionalized nucleotides are not digoxigenin labeled nucleotides. In particular, in some embodiments the functionalized nucleotides are not digoxigenin-11-dUTP.

In a preferred embodiment, the functionalized dNTPs are nucleotides comprising an alkyne group or a vinyl group, e.g. a modified nucleobase containing an alkyne (e.g. ethynyl) group or vinyl group, e.g. a nucleotide comprising a pyrimidine with an alkyne or vinyl group at position C5. In another preferred embodiment, the functionalized dNTPs are nucleotides comprising an azide group, e.g. comprising a modified sugar containing an azide group, e.g. a nucleotide comprising an azide group at position 2 of the deoxyribose sugar.

The reaction mixture for the RCA reaction must contain a combination of components capable of generating a RCA product from the template circular DNA molecule. For instance, the nucleotides present in the reaction mixture (e.g. the mixture of conventional and functionalized nucleotides) must be capable of hybridizing to their respective nucleotides in the circular DNA template to permit rolling circle amplification. The relative amounts of functionalized and conventional nucleotides present in the reaction mixture may vary depending on the identity of the functionalized nucleotide and the polymerase. Moreover, the relative amount of each nucleotide present in the reaction mixture may be used to control the incorporation of functionalized nucleotides into the RCA product. For instance, increasing the concentration of the functionalized nucleotides (or decreasing the proportion of conventional nucleotides) may result in a greater proportion of functionalized nucleotides in the RCA product (e.g. when the functionalized nucleotide is used in combination with its equivalent conventional nucleotide). Conversely, decreasing the concentration of the functionalized nucleotides (or increasing the proportion of conventional nucleotides) may result in a lower proportion of functionalized nucleotides in the RCA product.

Thus, the functionalized nucleotides may be used in addition to, or entirely in place of, the conventional nucleotides which hybridize to the same DNA base (nucleotide) in the template DNA. In some embodiments, the reaction mixture may contain only one type of functionalized nucleotide. In some embodiments, a combination of different functionalized nucleotides may be used in the same reaction. In some embodiments, all of the functionalized nucleotides in the reaction mixture contain the same type of functional group, e.g. alkyne group. In some embodiments, the functionalized nucleotides in the reaction mixture contain the different types of functional group. By way of example, the different types of functionalized nucleotides may be capable of hybridizing to the same DNA base (nucleotide), such as a dATP nucleotide functionalized with a fluorophore and a second dATP nucleotide functionalized with a sterol group. In another representative example, the different types of functionalized nucleotides may be capable of hybridizing to different DNA bases (nucleotides), such as a dATP nucleotide functionalized with a fluorophore and a dTTP nucleotide functionalized with a sterol group (or a different fluorophore to the fluorophore on the dATP nucleotide). Thus, any combination of functionalized and conventional nucleotides may be used in the invention. In a preferred embodiment, the RCA reaction mixture contains only one type of functionalized nucleotide.

The amount of functionalized nucleotides present in the reaction mixture can be measured as a relative percentage of the total nucleotides capable of hybridizing to a particular DNA base (nucleotide). Alternatively, this value can be considered as the percentage to which the functionalized nucleotide has replaced the corresponding conventional nucleotide. For example, using equal amounts of conventional dATP and a dATP modified with a fluorophore could be expressed as 50% of the total dATP nucleotides being modified (functionalized), or 50% replacement of the conventional dATP nucleotides with functionalized dATP nucleotides.

The relative amount of functionalized nucleotides in the RCA reaction mixture can be varied in order to control the frequency of functionalized nucleotides in the final single stranded oligonucleotides. In some embodiments, the functionalized nucleotides may represent up to about 5% of the total nucleotides capable of hybridizing to a particular DNA base (nucleotide), for example about 1%, 2%, 3%, 4% or 5%. Alternatively, in some embodiments the functionalized nucleotide may represent a higher proportion, such as 25%, 50%, 75% or 100% of the total nucleotides capable of binding to a particular DNA base (nucleotide).

The relative amount of functionalized nucleotides present may be varied for numerous reasons. For instance, some functionalized nucleotides, such as dATP modified with Cy3 are not available commercially at high concentrations. Moreover, as shown in the Examples, the inventors have determined that the inclusion of functionalized nucleotides may impact the yield of functionalized single stranded oligonucleotides produced by the invention, e.g. the use of high relative amounts of the functionalized nucleotide can inhibit the activity of the DNA polymerase responsible for the RCA reaction (e.g. reduce the efficiency at which the RCA product is synthesized), or the cleavage enzyme responsible for cleaving the RCA product and releasing the single stranded functionalized oligonucleotides (e.g. reduce the efficiency at which the RCA product is cleaved).

Notably however, the inventors have surprisingly determined that high yields of functionalized single stranded oligonucleotides may be achieved even when using high relative amounts of functionalized oligonucleotides, e.g. up to about 75%, such as up to about 70%, 65%, 60%, 55% or 50%. In some embodiments, it may be possible to use more than 75% of the functionalized oligonucleotides, such as about 80%, 85%, 90%, 95% or 100%. In particular, the inventors have unexpectedly found that functionalized nucleotides containing alkyne or vinyl groups in the nucleobase are particularly useful in the invention as they may be incorporated into the RCA product efficiently. Moreover, the RCA product could readily be cleaved to yield the functionalized single stranded oligonucleotides, although as discussed below, in some embodiments a higher amount of cleavage enzyme may be required relative to the amount needed to cleavage an equivalent RCA product containing only conventional nucleotides.

Similarly, the inventors have found that functionalized nucleotides containing O-methyl groups in the deoxyribose sugar may completely substitute a conventional nucleotide in the RCA reaction and still yield RCA products. Furthermore, the inventors surprisingly determined that incorporation of these nucleotides into the RCA products does not affect formation of the cleavage domains (e.g. hairpin cleavage domains) or their enzymatic cleavage (e.g. with restriction endonucleases).

Accordingly, the relative amount of functionalized nucleotide that is present in the reaction mixture may be adjusted to optimize the yield of the desired functionalized single stranded oligonucleotides or to optimize the generation of the RCA product. Such modifications are within the purview of the skilled person based on the methods described in the Examples below. Thus, in some embodiments, the relative amount of functionalized nucleotides in the reaction mixture may be about 1-5%, 1-10%, 1-25%, 5-25%, 10-25%, 25-50%, 25-75%, 25-100%, 50-75%, 50-100%, or 75-100%.

In addition to the relative amount of functionalized nucleotides in the reaction mixture, the absolute amount of nucleotides (both conventional and functionalized nucleotides) following the generation of the RCA product in step (b), the method comprises a step of cleaving the RCA product at the cleavage domains to release the single stranded functionalized oligonucleotides. As discussed above, the step of cleaving the RCA product may be achieved by contacting the RCA product with a cleavage enzyme under suitable conditions to selectively cleave the RCA product in the cleavage domains.

The term “release” is used in the present context to refer to cleaving the RCA product at the cleavage domains bordering the oligonucleotide sequences so as to detach or separate the functionalized oligonucleotides from the cleavage domains. It is desirable that the release of a given functionalized oligonucleotide will involve cleavage at both cleavage domains bordering the oligonucleotide sequence.

It is not necessary for cleavage to occur at all of the cleavage domains in the RCA product in order to generate the functionalized single stranded oligonucleotides. As noted above, incorporation of functionalized nucleotides in the RCA product, particularly in the cleavage domains, may reduce the efficiency of the cleavage step. Nevertheless, cleavage of a portion of cleavage domains will result in the release of a portion of functionalized single stranded oligonucleotides. Thus, in some embodiments, the step of cleaving the RCA product results in cleavage of at least about 30% of the cleavage domains in the RCA product, e.g. at least about 35%, 40%, 45%, 50%, 60%, 70% or 80%. In some embodiments, the step of cleaving the RCA product results in cleavage of at least about 90% of the cleavage domains in the RCA product, e.g. 95% or more.

Alternatively viewed, in some embodiments, the step of cleaving the RCA product results in the release of at least about 30% of the functionalized single stranded oligonucleotides contained in the RCA product, e.g. at least about 35%, 40%, 45%, 50%, 60%, 70% or 80%. In some embodiments, the step of cleaving the RCA product results in the release of at least about 90% of the functionalized single stranded oligonucleotides contained in the RCA product, e.g. 95% or more.

Once the single stranded functionalized oligonucleotides have been released, it may be desirable to isolate, separate or purify the single stranded functionalized oligonucleotides from the cleavage reaction mixture (e.g. reaction components and/or degradation products such as cleavage domains, uncleaved RCA products etc.) and for use in other applications.

Thus, in some embodiments, the method of the present invention further comprises a step of isolating, separating or purifying the functionalized single stranded oligonucleotides. This isolation, separation or purification may be done by any suitable method known in the art.

In some embodiments, following the isolation, separation or purification step the functionalized single stranded oligonucleotides are preferably substantially free of any contaminating components derived from the materials or component used in the isolation procedure or in their preparation (e.g. reaction components and/or degradation products such as cleavage domains, uncleaved RCA products etc.). In some embodiments, the functionalized single stranded oligonucleotides are purified to a degree of purity of more than about 50 or 60%, e.g. more than about 70, 80 or 90%, such as more than about 95 or 99% purity as assessed w/w (dry weight). Such purity levels may include degradation products of the functionalized single stranded oligonucleotides.

In some embodiments, it may be useful to prepare enriched preparations of the functionalized single stranded oligonucleotides which have lower purity, e.g. contain less than about 50% of the functionalized single stranded oligonucleotides of interest, e.g. less than about 40 or 30%.

As discussed above, the invention may result in a mixture or plurality (e.g. a library) of functionalized single stranded oligonucleotides. Thus, in some embodiments, it may be desirable to further separate the functionalized single stranded oligonucleotides, e.g. by size, to obtain specific functionalized single stranded oligonucleotides (i.e. to isolate specific functionalized single stranded oligonucleotides) or to generate sub-groups or sub-libraries of functionalized single stranded oligonucleotides. Any suitable means for separating the mixtures of functionalized single stranded oligonucleotides to isolate the specific functionalized single stranded oligonucleotides or sub-groups or sub-libraries of functionalized single stranded oligonucleotides may be employed.

Thus, in some embodiments, the method comprises a further step of separating functionalized single stranded oligonucleotides from a mixture (e.g. library) of functionalized single stranded oligonucleotides obtained by the method described above, to isolate a specific functionalized single stranded oligonucleotide or a sub-group of functionalized single stranded oligonucleotides.

For example, the products of the cleavage reaction may be separated by size using gel electrophoresis using an agarose gel or a polyacrylamide gel. The desired functionalized oligonucleotides can then be isolated from the gel and purified further, if necessary, according to methods known in the art. Other methods for purifying, isolating or separating the functionalized oligonucleotides of the invention utilize chromatography (e.g. HPLC, size-exclusion, ion-exchange, affinity, hydrophobic interaction, reverse-phase) or capillary electrophoresis.

As mentioned above, the functionalized oligonucleotides produced by the method described above may contain a reactive group that is capable of reacting with another chemical group, e.g. a chemical group on a molecule or component to be conjugated to the functionalized oligonucleotide, e.g. via click chemistry. For instance, conjugating additional molecules or components (which themselves may comprise or be viewed as functional groups) to the single stranded oligonucleotides may be particularly useful for incorporating large or bulky groups, such as groups that may inhibit or lower the efficiency of the present method if present in the functionalized nucleotides used in the RCA reaction. Thus, for example, functionalized oligonucleotides may be produced that contain molecules or components that could not be incorporated by a polymerase directly during the RCA reaction, or would only be incorporated at low efficiencies or yields. Additionally or alternatively, subjecting the functionalized oligonucleotides obtained by the method to a further conjugation step may increase the diversity of the structures present in a functionalized oligonucleotide library.

Accordingly, in some embodiments, the method further comprises a step of conjugating a molecule or component to the functionalized oligonucleotide(s) via a functional (e.g. reactive) group in the oligonucleotide, such as via click chemistry. In some preferred embodiments, the molecule or component is conjugated to the functionalized oligonucleotide via an alkyne, vinyl or azide group (i.e. an alkyne, vinyl or azide group in a functionalized nucleotide incorporated into the RCA product in the method defined herein).

The term “conjugation” in the context of the present invention with respect to linking or joining a molecule or component to a functionalized oligonucleotide (e.g. an alkyne, vinyl or azide group in said functionalized oligonucleotide) refers to joining said molecule or component to said oligonucleotide via a covalent bond. In particular, this conjugation may occur via a click chemistry reaction.

An example of a specific click chemistry reaction that may be used to conjugate additional molecules or components to single stranded functionalized oligonucleotides of the present invention comprising one or more alkyne groups is the azide-alkyne cycloaddition. In order to achieve the desired conjugation, the oligonucleotide comprising the alkyne group may be incubated for a suitable period of time with a molecule or component (e.g. a label such as a fluorophore) containing an azide group. The azide-alkyne cycloaddition reaction commonly uses a copper catalyst, particularly a copper(I) catalyst. In some embodiments, the oligonucleotide and the azide-containing molecule or component may be incubated in the presence of copper sulfate. A reducing agent may also be used to generate the active copper(I) catalyst. The reducing agent may be, for example, sodium ascorbate.

A further representative example for conjugating additional molecules or components to single stranded functionalized oligonucleotides of the present invention comprising one or more vinyl groups may utilize the alkene-tetrazine reaction. This reaction has the advantage of being copper free. Moreover, it is entirely orthogonal to the aforementioned alkyne-azide click chemistry reaction. Accordingly, a single stranded functionalized oligonucleotide comprising both an alkyne group and a vinyl group could participate in two click chemistry reactions independently to conjugate two different additional molecules or components to the same oligonucleotide.

Thus, in some embodiments, the invention may be seen as providing a two-step method for producing functionalized single stranded oligonucleotides comprising a first step of incorporating functionalized nucleotides (e.g. comprising reactive groups, such as groups capable of participating in click chemistry reactions, e.g. alkyne, vinyl or azide groups) into an oligonucleotide using the method described herein, and a second step of conjugating additional molecules or components to the single stranded oligonucleotides via the functional groups in the functionalized nucleotides.

It will be evident that any desirable molecules or components (i.e. entities) may be conjugated to the functional groups in the single stranded oligonucleotides produced by the present method. Such molecules or components simply require the presence of a group (e.g. reactive group) capable of reacting with a functional group in the oligonucleotide to form a covalent bond. In some embodiments, the molecule or component may be a nucleic acid molecule, protein, peptide, small-molecule organic compound (e.g. a sterol, such as cholesterol), fluorophore, metal-ligand complex, polysaccharide, nanoparticle, nanotube, polymer, cell, organelle, vesicle, virus, virus-like particle or any combination of these.

The cell may be a prokaryotic or eukaryotic cell. In some embodiments the cell is a prokaryotic cell, e.g. a bacterial cell.

In some embodiments, the functionalized oligonucleotide may be conjugated or fused to a compound or molecule which has a therapeutic or prophylactic effect, e.g. an antibiotic, antiviral, vaccine, antitumour agent, e.g. a radioactive compound or isotope, cytokine, toxin, oligonucleotide, nucleic acid encoding gene or nucleic acid vaccine.

In some embodiments, the functionalized oligonucleotide (e.g. an aptamer) may be conjugated or fused to a label, e.g. a radiolabel, a fluorescent label, luminescent label, a chromophore label as well as to substances and enzymes which generate a detectable substrate, e.g. horse radish peroxidase, luciferase or alkaline phosphatase. This detection may be applied in numerous assays where antibodies are conventionally used, including Western blotting/immunoblotting, histochemistry, enzyme-linked immunosorbent assay (ELISA), or flow cytometry (FACS) formats. Labels for magnetic resonance imaging, positron emission tomography probes and boron 10 for neutron capture therapy may also be conjugated to the functionalized oligonucleotides described herein.

In some embodiments, the molecule or component may be selected from the group consisting of: a fluorophore, a sterol (e.g. cholesterol), a polyether, a metal complex, a thiol containing molecule, a molecule containing a group providing increased nuclease resistance and a molecule containing a group capable of participating in a click chemistry reaction.

It will be evident that the molecule or component conjugated to the functionalized oligonucleotide may interact with other molecules and such interactions may be covalent or non-covalent interactions. For instance, a peptide conjugated to the oligonucleotide may interact with its cognate binding partner, such as an antibody, non-covalently. In a further example, a molecule containing a group capable of participating in a click chemistry reaction may by conjugated to another molecule or component as defined above via reaction with a reactive group of said molecule or component to form a covalent complex.

The circular DNA molecule provided in step (a) of the present method may be produced by any suitable means and a variety of means are well-known in the art. Accordingly, the method of the present invention may comprise additional steps before step (a) of providing a circular DNA molecule.

For instance, the process of producing the circular DNA molecule may comprise a step of designing a sequence comprising one or more desired oligonucleotide sequences bordered by cleavage domains (termed a “pseudogene” herein) in silico. Thus, the method may utilize a computer-implemented method of designing the pseudogene and such methods are described in the art, e.g. Ducani et al., 2013, supra.

The term “pseudogene” as used herein refers to a nucleotide sequence comprising one or more desired oligonucleotide sequences, bordered by cleavage domains. Alternatively, this sequence may be referred to herein as the “nucleic acid construct”.

The pseudogene sequence designed in silico can be produced (e.g. synthesized) using commercially available gene synthesis methods, or any other suitable means known in the art, e.g. assembly PCR. Thus, the method may comprise a step of producing the pseudogene.

The pseudogene may be produced as a single stranded or double stranded molecule. The pseudogene may circularized using any suitable means known in the art to provide the circular DNA molecule of step (a). For instance, a double stranded molecule may be ligated using a ligase enzyme, as described below. It will be understood in this regard that at least one of the 5′ ends of the pseudogene is phosphorylated to enable ligation to take place. In embodiments where the step of producing the pseudogene results in a DNA molecule in which the 5′ ends are not phosphorylated, the method may comprise a further step of phosphorylating the 5′ end(s) of the pseudogene, e.g. using a kinase enzyme, such as T4 polynucleotide kinase.

Similarly, where the pseudogene is provided as a single stranded molecule, it may be circularized using an appropriate ligase enzyme capable of catalyzing intramolecular ligation of single stranded oligonucleotide molecules, such as CircLigase. Again, the method may comprise a further step of phosphorylating the 5′ end(s) of the pseudogene, e.g. using a kinase enzyme, such as T4 polynucleotide kinase, to facilitate the ligation reaction.

In some embodiments it may be advantageous to insert the pseudogene into a plasmid, which can be replicated in bacteria, such as E. coli. Suitable plasmid sequences are well-known in the art. This allows the sequence of the pseudogene to be checked (e.g. by sequencing) and for any errors in the sequence to be corrected, e.g. via iterations of sequencing and mutagenesis using any suitable methods.

The insertion of the pseudogene into a plasmid also facilitates the generation of a significant number of copies of the circular DNA molecule. For instance, a single bacterial colony containing a plasmid comprising the sequence-verified pseudogene may be grown to amplify the plasmid, which may be subsequently purified from the bacteria using any suitable means known in the art.

The pseudogene sequence is then excised from the plasmid, e.g. using restriction enzymes as described above. The excised linear pseudogene sequence may be purified using PAGE and gel extraction, or other suitable methods. The purified linear pseudogene sequence can then be re-circularized using a suitable ligase enzyme, such as T4 ligase, to form the circular DNA molecule to be provided in step (a) of the present method.

In addition to amplification, the process of transfecting the pseudogene containing plasmid into bacteria also allows a bacterial glycerol stock to be produced. Bacteria comprising the desired pseudogene plasmid can be prepared in glycerol, frozen and stored stably for long periods of time.

Accordingly, in some embodiments step (a) of the present method comprises:

(i) cloning into a DNA plasmid a linear DNA molecule comprising the oligonucleotide sequence bordered by cleavage domains;

(ii) amplifying said plasmid;

(iii) excising part of the plasmid containing the DNA molecule comprising the oligonucleotide sequence bordered by cleavage domains; and

(iv) circularizing the part of the plasmid obtained in step (iii).

In some embodiments, step (ii) comprises transfecting said DNA plasmid into bacteria and growing the bacteria.

In some embodiments, the linear DNA molecule comprising the oligonucleotide sequence bordered by cleavage domains further comprises a 5′ end region and a 3′ end region each comprising a cleavage domain and wherein step (iii) comprises cleaving the cleavage domains in the end regions with a cleavage enzyme.

Any suitable cleavage enzyme as defined herein may be used to excise the pseudogene from the plasmid. In some embodiments, the cleavage enzyme is BsmBI or BsaI or an isoschizomer thereof.

In some particular embodiments of the invention, the method uses a phi29 DNA polymerase or a derivative thereof and a functionalized nucleotide comprising a modified nucleobase. In some embodiments, the functionalized nucleotide comprises a reactive group in the nucleobase, such as a reactive group capable of participating in a click chemistry reaction. In some embodiments, the reactive group in the nucleobase is an alkyne or a vinyl group as defined above. In some embodiments, the relative amount of functionalized nucleotide in the RCA mixture is about 25-100%, e.g. 25-75%, 50-100% or 75-100% or any value within these ranges. In some embodiments, the cleavage domains are capable of forming hairpin structures in the RCA product as defined above.

In a further aspect, the present invention provides a kit, particularly a kit for use in producing functionalized single stranded oligonucleotides, said kit comprising:

(i) a circular DNA molecule comprising an oligonucleotide sequence bordered by cleavage domains; and optionally

(ii) one or more cleavage enzymes that cleave the cleavage domains of (i); and/or

(iii) functionalized dNTPs.

In some embodiments, the invention provides kit for use in the method described herein comprising:

(i) a circular DNA molecule comprising an oligonucleotide sequence bordered by cleavage domains, wherein the cleavage domains comprise or consist of a sequence capable of forming a hairpin structure and wherein the double-stranded portion of the hairpin structure comprises a sequence that is recognized by a cleavage enzyme; and

(ii) functionalized dNTPs (e.g. as defined herein); and optionally

(iii) one or more cleavage enzymes that cleave the cleavage domains of (i).

In some embodiments, the kit may comprise a DNA polymerase enzyme capable of performing rolling circle amplification, i.e. comprising at least some strand displacement activity, as defined herein. For instance, the kit may comprise a phi29 polymerase or derivative thereof, or a Bst DNA polymerase or derivative thereof.

In some embodiments, the kit may comprise one or more nickases. For instance, the kit may comprise Nb.BsrDI, Nt.BspQI or a combination thereof.

In some embodiments, the kit may comprise one or more single stranded binding protein as defined herein. For instance, the kit may comprise E. coli single stranded DNA binding protein, gene 32 protein of T4 phage, or a combination thereof.

In some embodiments, the kit may comprise one or more molecules or components to be conjugated to a functionalized single stranded oligonucleotide as defined above.

The circular DNA molecule, cleavage enzymes and functionalized dNTPs of the kit are as described above.

In a further aspect, the present invention provides a single stranded functionalized oligonucleotide obtained by the method as described herein.

In some embodiments, the single stranded functionalized oligonucleotide contains at least 20 nucleotides. For instance, the single stranded functionalized oligonucleotide may contain at least 25, 30, 35, 40, 45, 50, 60, 70, 80, 90, 100 or more nucleotides. In some preferred embodiments, the single stranded functionalized oligonucleotide contains at least 50 nucleotides, e.g. at least about 55, 60, 65, 70, 75, 80, 85, 90, 95 or 100 nucleotides. In some embodiments, the single stranded functionalized oligonucleotide contains about 50-1000, 55-900, 60-800, 65-700, 70-600, 80-500, 90-450 or 100-400 nucleotides. In some embodiments, the single stranded functionalized oligonucleotide contains about 400-10000, 500-9000, 600-8000, 700-7000, 800-6000, 900-5000 or 1000-4000 nucleotides, e.g. comprising about 500, 1000, 1500, 2000, 2500, 3000, 3500 or more nucleotides.

In some embodiments, at least 5% of the nucleotide residues of the single stranded functionalized oligonucleotide are functionalized nucleotides, i.e. nucleotides containing a functional group as defined herein. In some embodiments, at least 10%, 15%, 20%, 25%, 30%, 35%, 40%, 45%, 50% or more of the nucleotide residues of the single stranded functionalized oligonucleotide are functionalized nucleotides. Thus, in some embodiments, about 5-100% of the nucleotide residues of the single stranded functionalized oligonucleotide are functionalized nucleotides, e.g. about 10-95%, 15-90%, 20-85%, 25-80%, 30-75%, 35-70%, 40-65% or 45-55% of the nucleotide residues of the single stranded functionalized oligonucleotide are functionalized nucleotides.

In some embodiments, the single stranded functionalized oligonucleotide contains at least one internal functionalized nucleotide, i.e. a functionalized nucleotide that is not at the 5′ or 3′ end of the oligonucleotide. In some embodiments, the single stranded functionalized oligonucleotide contains 2, 3, 4, 5, 6, 7, 8, 9, 10 or more internal functionalized nucleotides, e.g. 15, 20, 25, 30, 35, 40, 45, 50 or more internal functionalized nucleotides.

The single stranded functionalized oligonucleotide may contain any one or more of the functionalized nucleotides defined herein. In some embodiments, the stranded functionalized oligonucleotide comprises functionalized nucleotides containing a functional group selected from an alkyne group, an alkene group (e.g. a vinyl group), an azide group, a halogen group, an O-methyl group, a locked ribose sugar or a combination thereof.

Thus, in one embodiment, the invention provides a single stranded functionalized oligonucleotide obtained by the method described herein, wherein;

(i) the oligonucleotide contains at least 50 nucleotides; and

(ii) at least 5% of the nucleotide residues contain a functional group selected from an alkyne group, an alkene group (e.g. vinyl), an azide group, a halogen group, an O-methyl group, a locked ribose sugar or a combination thereof, wherein the preferred positions of the functional groups within the functionalized nucleotide residues are as defined above.

In a yet further aspect, the present invention provides a library comprising a plurality of single stranded functionalized oligonucleotides obtained by the method as described herein. The library of a plurality of single stranded functionalized oligonucleotides may include one or more single stranded functionalized oligonucleotides as defined above.

In yet a further aspect, the present invention provides a method as defined herein being a method for producing a pool of single stranded functionalized oligonucleotides for use in single molecule fluorescence in situ hybridization (smFISH), wherein the functionalized nucleotides are fluorescently labeled nucleotides and wherein each single stranded functionalized oligonucleotide in the pool contains about 15-30, preferably about 20-25 nucleotides. In preferred embodiments, the functionalized nucleotides are labeled with the same fluorescent molecule.

As an alternative to the use of directly fluorescently labeled functionalized nucleotides, the method may involve the use of nucleotides comprising a chemical group capable of participating in click chemistry, to which a fluorescent label can subsequently be conjugated. Suitable fluorescent labels are well known in the art and are defined above.

Accordingly, in yet a further aspect, the present invention provides a method as defined herein being a method for producing a pool of single stranded functionalized oligonucleotides for use in single molecule fluorescence in situ hybridization (smFISH), wherein the functionalized nucleotides are nucleotides comprising a chemical group capable of participating in click chemistry, and wherein the method further comprises a step of conjugating a fluorescent label to at least one functionalized nucleotide in each functionalized oligonucleotide via click chemistry, and wherein each single stranded functionalized oligonucleotide in the pool contains about 15-30, preferably about 20-25, nucleotides. Nucleotides comprising a chemical group capable of participating in click chemistry are defined above, and include nucleotides comprising an azide group, an alkyne group, an alkene group, a nitrone group, a tetrazine group or a tetrazole group, or a combination thereof.

In the smFISH technique, the single stranded functionalized oligonucleotides act as hybridization probes to identify the presence and location of nucleic acid molecules which comprise a specific target sequence to be detected. Accordingly, once the pool of single stranded functionalized oligonucleotides for use in single molecule fluorescence in situ hybridization (smFISH) has been produced by the method disclosed herein, the functionalized oligonucleotides may be hybridized to a nucleic acid molecule comprising a target sequence to be detected in an smFISH method.

Where the single stranded functionalized oligonucleotides comprise functionalized nucleotides comprising a chemical group capable of participating in click chemistry, the step of conjugating a fluorescent label to the functionalized nucleotides via click chemistry may be carried out before or after the functionalized oligonucleotides are hybridized to a nucleic acid molecule comprising a target sequence.

For instance, the functionalized oligonucleotides comprising functionalized nucleotides comprising a chemical group capable of participating in click chemistry may be subjected to a step of conjugating a fluorescent label to at least one functionalized nucleotide in each functionalized oligonucleotide via click chemistry, and may then be hybridized to a nucleic acid molecule comprising a target sequence, once the fluorescent labels have been conjugated. Alternatively, the functionalized oligonucleotides comprising functionalized nucleotides comprising a chemical group capable of participating in click chemistry may first be hybridized to a nucleic acid molecule comprising a target sequence, and may then be subjected to a step of conjugating a fluorescent label to at least one functionalized nucleotide in each functionalized oligonucleotide via click chemistry, once hybridized to the target sequence.

Accordingly, in a yet further aspect, the present invention provides a method as defined herein being a method for producing a pool of single stranded functionalized oligonucleotides for use in single molecule fluorescence in situ hybridization (smFISH), wherein the functionalized nucleotides are nucleotides comprising a chemical group capable of participating in click chemistry, and wherein the method further comprises a step of conjugating a fluorescent label to at least one functionalized nucleotide in each functionalized oligonucleotide via click chemistry, and wherein each single stranded functionalized oligonucleotide in the pool contains about 15-30, preferably about 20-25, nucleotides, and wherein the step of conjugating a fluorescent label to at least one functionalized nucleotide in each functionalized oligonucleotide via click chemistry is carried out before the functionalized oligonucleotides are hybridized to a nucleic acid molecule comprising a target sequence.

Similarly, the present invention also provides a method as defined herein being a method for producing a pool of single stranded functionalized oligonucleotides for use in single molecule fluorescence in situ hybridization (smFISH), wherein the functionalized nucleotides are nucleotides comprising a chemical group capable of participating in click chemistry, and wherein the method further comprises a step of conjugating a fluorescent label to at least one functionalized nucleotide in each functionalized oligonucleotide via click chemistry, and wherein each single stranded functionalized oligonucleotide in the pool contains about 15-30, preferably about 20-25, nucleotides, and wherein the step of conjugating a fluorescent label to at least one functionalized nucleotide in each functionalized oligonucleotide via click chemistry is carried out after the functionalized oligonucleotides are hybridized to a nucleic acid molecule comprising a target sequence.

A “pool” of single stranded functionalized oligonucleotides refers to a plurality of oligonucleotides with different sequences, i.e. sequences that hybridize to different (e.g. non-overlapping) sequences within the target (the molecule to be detected). In some embodiments, the pool contains at least about 20 oligonucleotides, e.g. about 22, 25, 27, 30, 35, 40, 45, 50, 60, 70, 80, 90 or 100 oligonucleotides. For instance, in some embodiments, the pool contains about 30-96 oligonucleotides, e.g. about 36, 48, 60, 72, 84 or 96 oligonucleotides.

Alternatively viewed, in some embodiments the pseudogene contains at least about 20 oligonucleotide sequences bordered by cleavage domains, e.g. about 22, 25, 27, 30, 35, 40, 45, 50, 60, 70, 80, 90 or 100 oligonucleotide sequences bordered by cleavage domains. For instance, in some embodiments, the pseudogene contains about 30-96 oligonucleotide sequences bordered by cleavage domains, e.g. about 36, 48, 60, 72, 84 or 96 oligonucleotide sequences bordered by cleavage domains.

The invention will now be described in more detail in the following non-limiting Examples with reference to the following drawings:

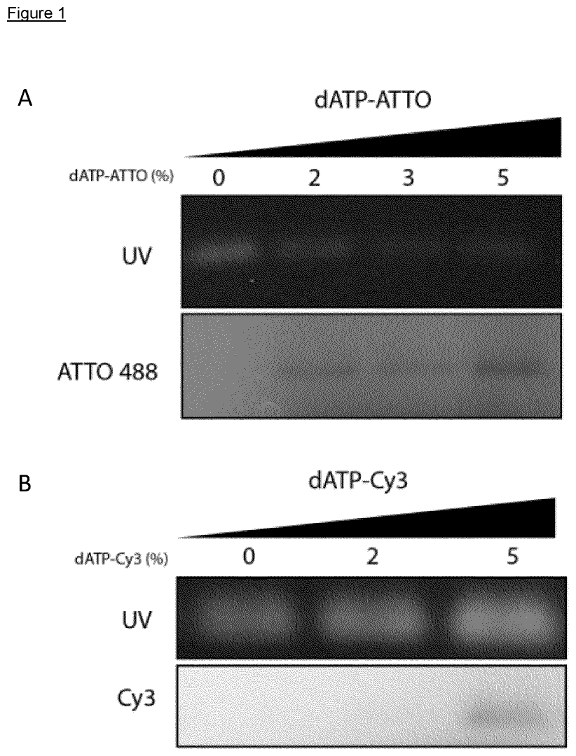

FIG. 1 shows photographs of agarose gels visualized using UV light following ethidium bromide staining (top), and fluorescent imaging using wavelengths corresponding to the emission wavelengths of the fluorophores (bottom). The agarose gels show functionalized single stranded oligonucleotide products of the invention (containing 378 nucleotides) comprising fluorophores ATTO-488 (A) or Cy3 (B).

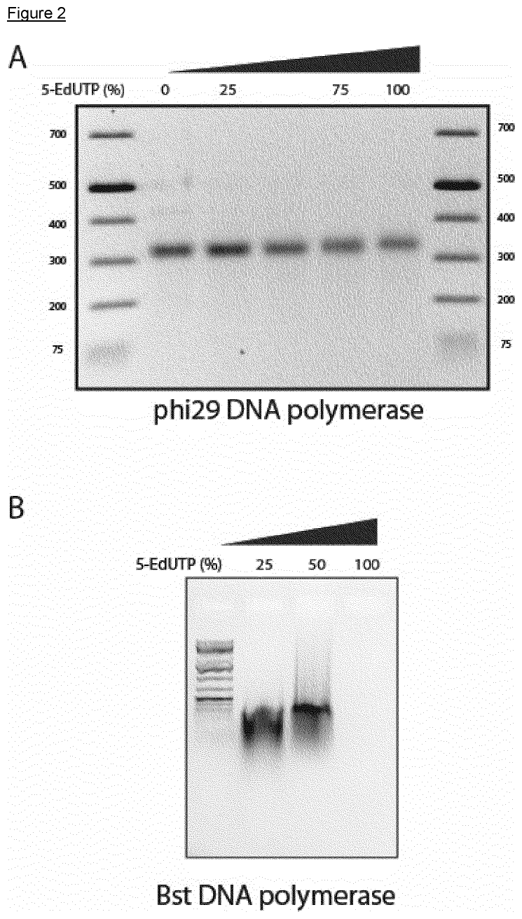

FIG. 2 shows the negative image of a photograph of an agarose gel visualized using UV light following ethidium bromide staining. The agarose gel shows functionalized single stranded oligonucleotide products of the invention (containing 420 nucleotides) comprising 5-ethnyl-dUTP (5-EdUTP) produced using various relative amounts of 5-EdUTP/dTTP nucleotides, i.e. 25%, 50%, 75% and 100% and phi29 DNA polymerase (A) or Bst DNA polymerase (B).

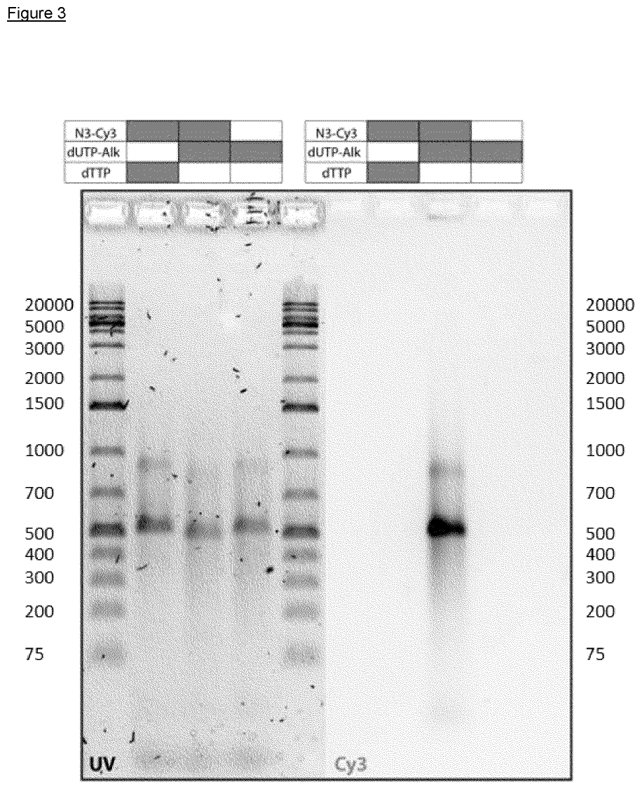

FIG. 3 shows a photograph of an agarose gel visualized using UV light following ethidium bromide staining (left), and fluorescent imaging using wavelengths corresponding to the emission wavelengths of the Cy3 fluorophores (right). The agarose gels show single stranded oligonucleotide products of the invention (containing 420 nucleotides) comprising 5-ethnyl-dUTP (5-EdUTP) or conventional dTTP that were subsequently reacted with Cy3 fluorophore-azide molecule (N3-Cy3). The shaded boxes denote the presence of the indicated species.

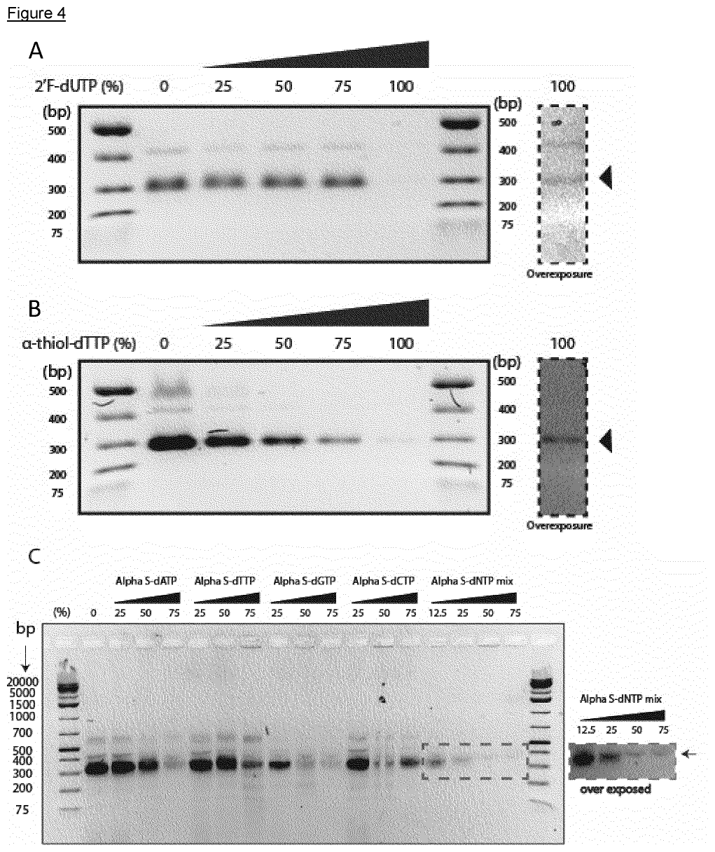

FIG. 4 shows negative images of photographs of agarose gels visualized using UV light following ethidium bromide staining. The agarose gels show functionalized single stranded oligonucleotide products of the invention (containing 420 nucleotides) comprising: (A) 2′-Fluoro-2′-deoxyuridine-5′-triphosphate (2′F-dUTP); (B) 2′-Deoxythymidine-5′-O-(1-Thiotriphosphate) (α-thiol-dTTP); and (C) 2-dNTP Alpha S nucleotides (Alpha S-dATP, Alpha S-dTTP, Alpha S-dCTP, Alpha S-dTTP, and an Alpha S-dNTP mixture), produced using various relative amounts of the functionalized nucleotides. The right panels in A and B show the lane corresponding to 100% functionalized nucleotides overexposed to show a band corresponding to the RCA product. The right panel in C show the lanes corresponding to Alpha S-dNTP mixture overexposed to show bands corresponding to the RCA product.

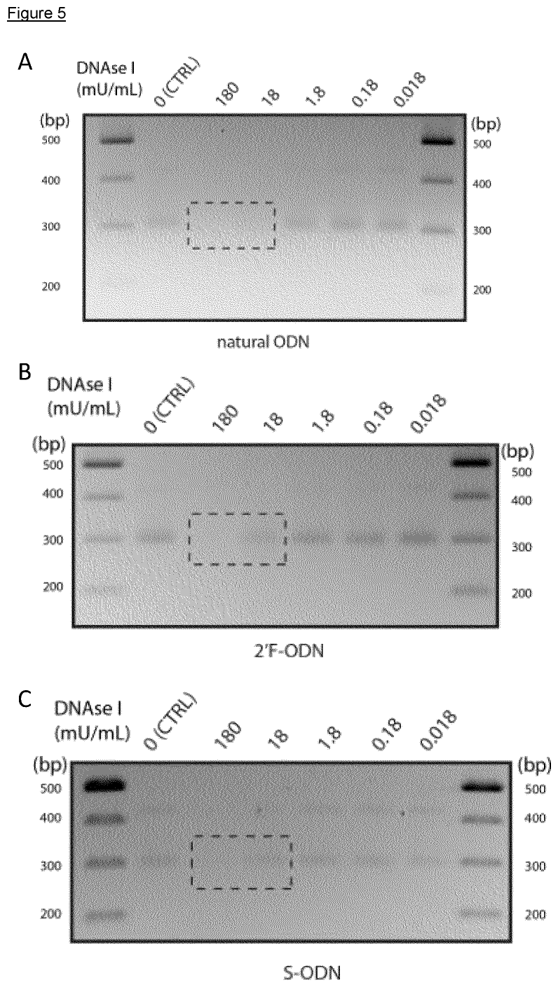

FIG. 5 shows negative images of photographs of agarose gels visualized using UV light following ethidium bromide staining. The agarose gels show oligonucleotides produced by the invention subjected to various concentrations of DNase I, wherein: (A) shows the reaction products of an oligonucleotide containing only conventional nucleotides (natural ODN); (B) shows the reaction products of an oligonucleotide containing 2′-Fluoro-2′-deoxyuridine-5′-triphosphate functionalized nucleotides (2′F-dUTP); and (C) shows the reaction products of an oligonucleotide containing 2′-Deoxythymidine-5′-O-(1-Thiotriphosphate) (S-ODN).

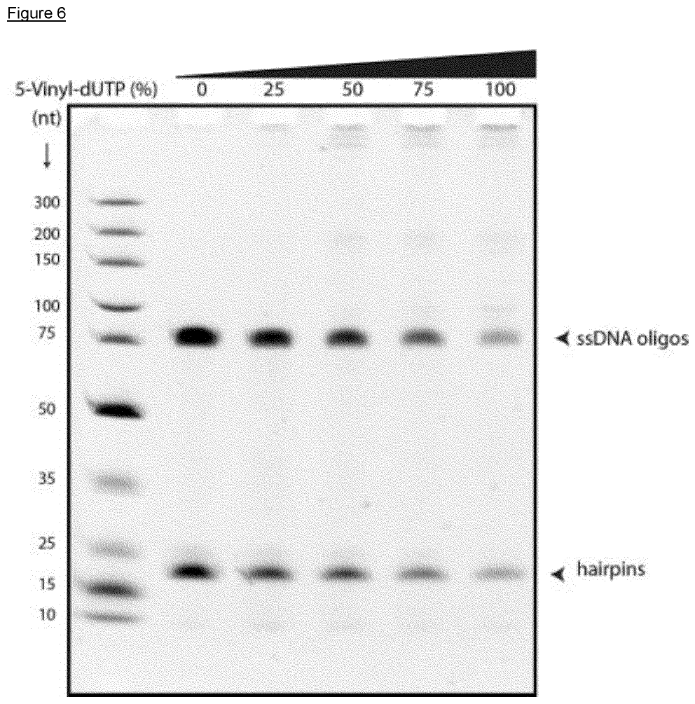

FIG. 6 shows a negative image of a photograph of a denaturing PAGE gel visualized using UV light following SybrGold staining. The PAGE gel shows functionalized single stranded oligonucleotide products of the invention comprising 5-Vinyl-2′-deoxyuridine-5′-triphosphate (5-Vinyl-dUTP) produced using various relative amounts of 5-Vinyl-dUTP nucleotides, i.e. 25%, 50%, 75% and 100%.

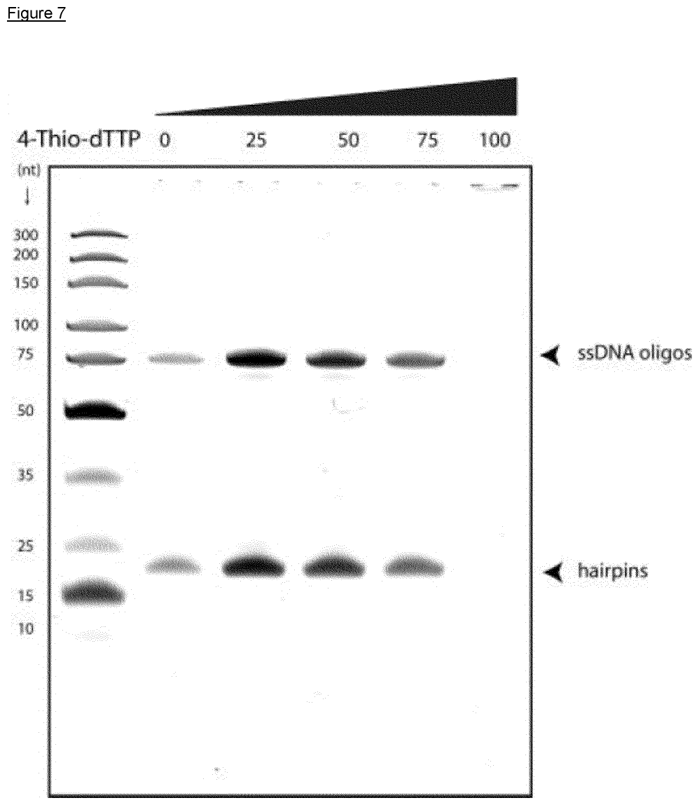

FIG. 7 shows a negative image of a photograph of a denaturing PAGE gel visualized using UV light following SybrGold staining. The PAGE gel shows functionalized single stranded oligonucleotide products of the invention comprising 4-Thiothymidine-5′-Triphosphate (4-Thio-dTTP) produced using various relative amounts of 5-Vinyl-dUTP nucleotides, i.e. 25%, 50%, 75% and 100%.

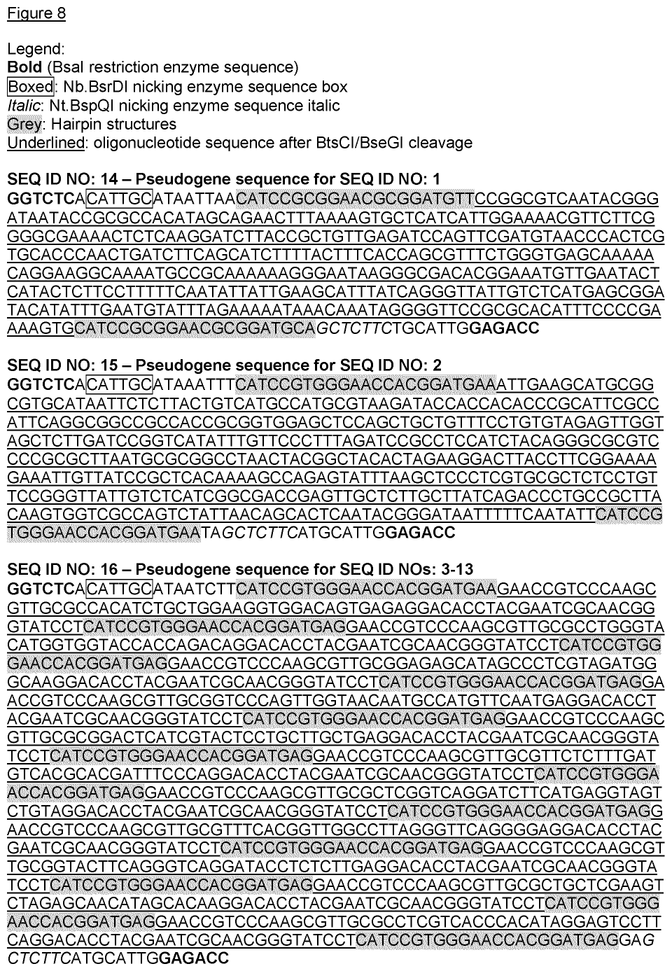

FIG. 8 shows annotated versions of the pseudogene sequences that were used in the production of oligonucleotides having sequences corresponding to SEQ ID NOs: 1-13. The sequences recognized by the cleavage and nicking enzymes, the hairpin sequences, and final oligonucleotide sequences are identified.

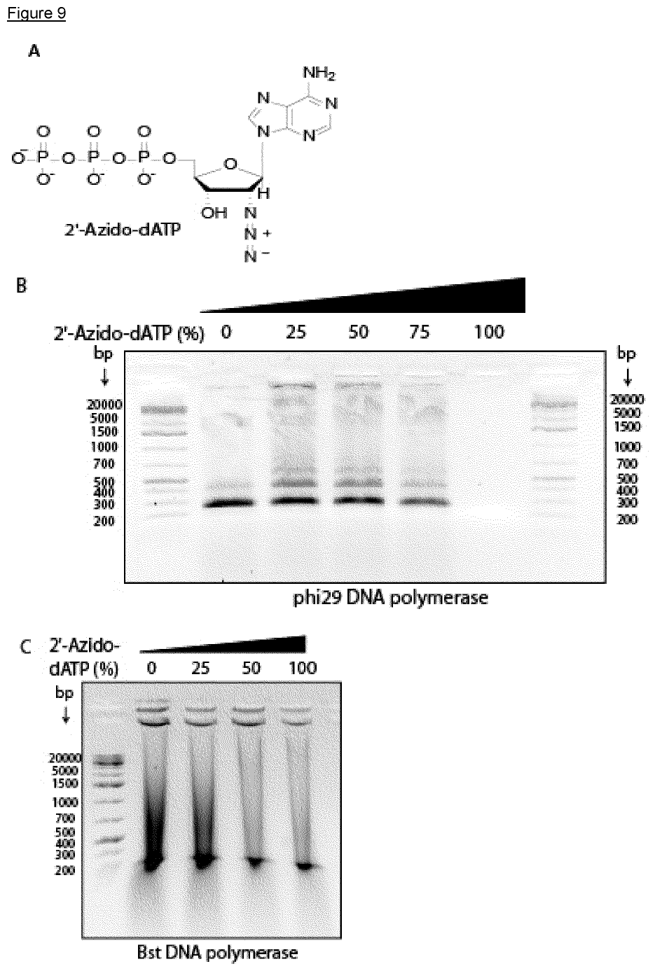

FIG. 9 shows the structure of a 2′-Azido-dATP (A) and negative images of photographs of denaturing PAGE gels visualized using UV light following SybrGold staining (B and C). The PAGE gels show functionalized single stranded oligonucleotide products of the invention comprising 2′-Azido-dATP produced using various relative amounts of 2′-Azido-dATP nucleotides, i.e. 25%, 50%, 75% and 100% and phi29 DNA polymerase (B) or Bst DNA polymerase (C).

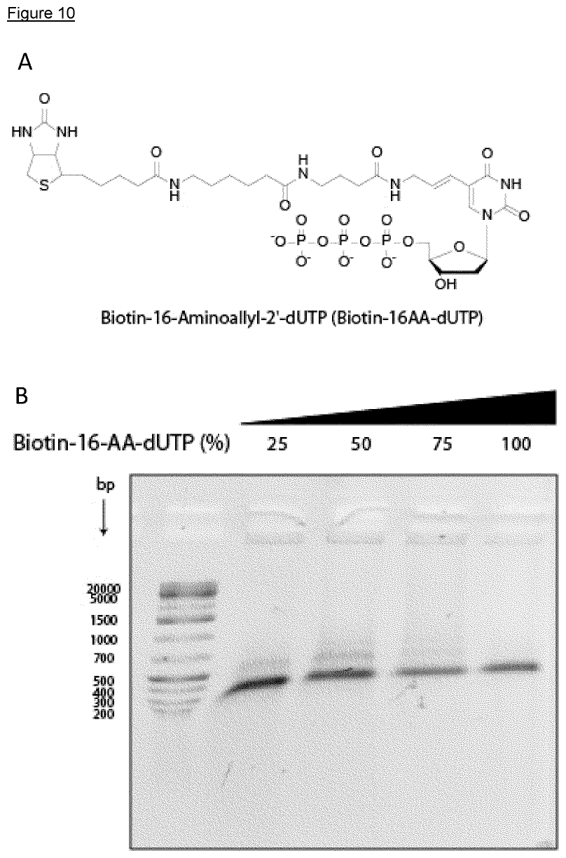

FIG. 10 shows the structure of a Biotin-16-Aminoallyl-2′-dUTP (A) and a negative image of a photograph of a denaturing PAGE gel visualized using UV light following SybrGold staining (B). The PAGE gel shows functionalized single stranded oligonucleotide products of the invention comprising Biotin-16-Aminoallyl-2′-dUTP produced using various relative amounts of Biotin-16-AA-dUTP nucleotides, i.e. 25%, 50%, 75% and 100% and phi29 DNA polymerase.

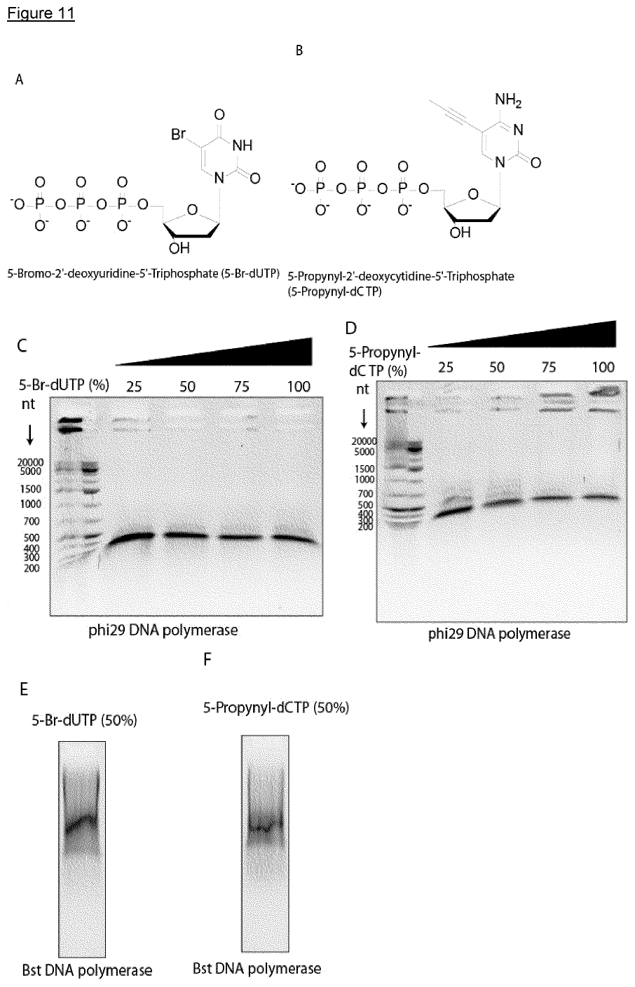

FIG. 11 shows the structures of a 5′-Bromo-2′-deoxyuridine-5′Triphosphate nucleotide (A) and a 5′-Propynyl-2′-deoxycytidine-5′-Triphosphate nucleotide (B); negative images of photographs of denaturing PAGE gels visualized using UV light following SybrGold staining (C, D, E and F). The PAGE gels show functionalized single stranded oligonucleotide products of the invention comprising 5′-Br-dUTP (C and E) or 5′-Propynyl-dCTP (D and F) produced using various relative amounts of the respective functionalized nucleotides, i.e. 25%, 50%, 75% and 100% and phi29 DNA polymerase or Bst DNA polymerase.

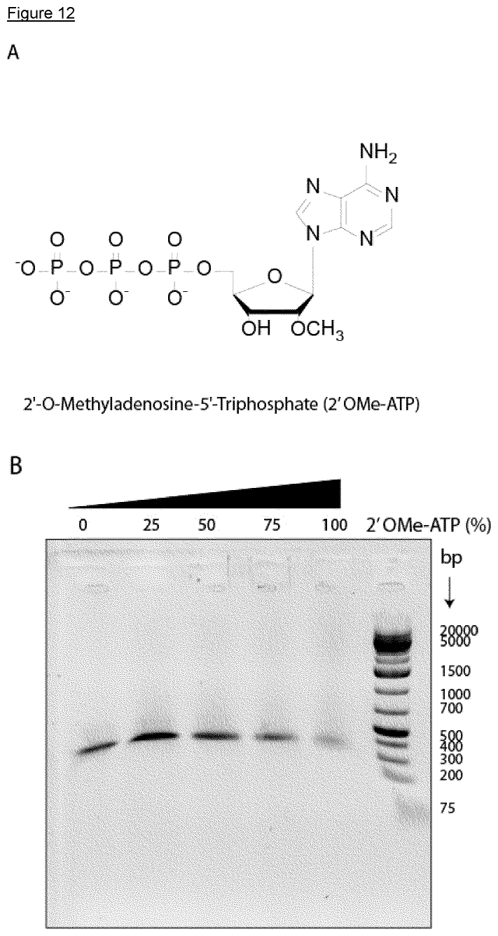

FIG. 12 shows the structure of a 2′-O-Methyladenosine-5′-Triphosphate nucleotide (A) and a negative image of a photograph of a denaturing PAGE gel visualized using UV light following SybrGold staining (B). The PAGE gel shows functionalized single stranded oligonucleotide products of the invention comprising 2′-OMe-ATP produced using various relative amounts of 2′-OMe-ATP nucleotides, i.e. 25%, 50%, 75% and 100% and phi29 DNA polymerase.

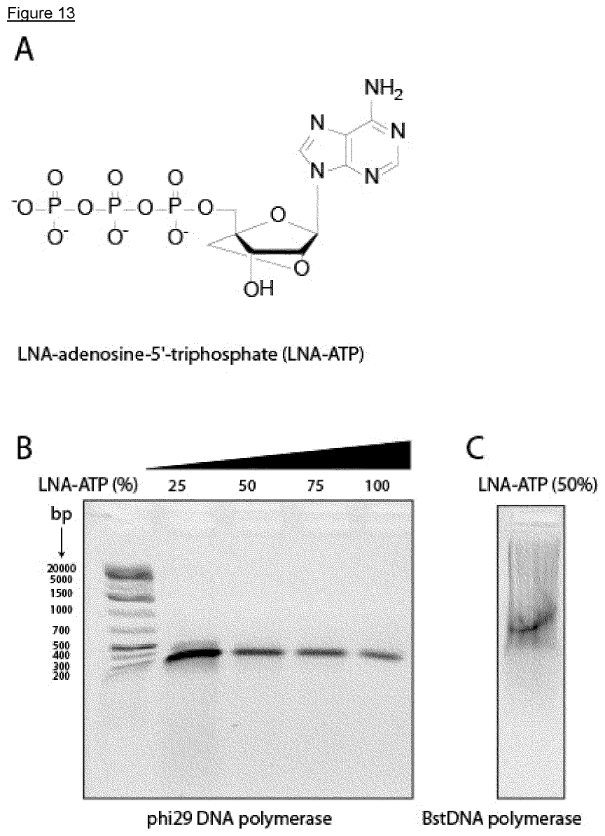

FIG. 13 shows the structure of an LNA-adenosine-5′-triphosphate nucleotide (A) and negative images of photographs of denaturing PAGE gels visualized using UV light following SybrGold staining (B and C). The PAGE gels show functionalized single stranded oligonucleotide products of the invention comprising LNA-ATP produced using various relative amounts of LNA-ATP nucleotides, i.e. 25%, 50%, 75% and 100% and phi29 DNA polymerase (B) or Bst DNA polymerase (C).

### EXAMPLES

### Example 1—Enzymatic Production of Single Stranded Oligonucleotides Comprising Fluorescent Nucleotides

Single stranded fluorescent oligonucleotides 378 nucleotides in length (SEQ ID NO: 1) were produced enzymatically using phi29 DNA polymerase. This was done via incorporation of two different functionalized dATP nucleobases, one comprising the fluorophore Cy3 (7-Propargylamino-7-deaza-ATP-Cy3) and one comprising the fluorophore ATTO-488 (7-Propargylamino-7-deaza-ATP-ATTO-488).

A double stranded circular DNA template containing SEQ ID NO: 1 and hairpin cleavage domains was prepared as described in Ducani et al., 2013, Nature Methods, 647-652. The template (1 ng/μL) was nicked with Nb.BsrDI and Nt.BspQI (0.25 U/μL) and a rolling circle amplification reaction (0.1-0.25 ng/μL template DNA, phi29 DNA polymerase 0.25 U/μL, 0.1 μg T4 gene 32) was performed several times with different ratios of natural dATPs and functionalized dATPs in each reaction (i.e. different relative amounts of the functionalized dATP, 2%, 3% or 5%). The resulting RCA products were diluted five times in deionized water and 1× digestion buffer (50 mM Potassium Acetate, 20 mM Tris-acetate, 10 mM Magnesium Acetate, 100 μg/ml BSA, pH 7.9 at 25° C.) and were then digested with BtsCI restriction enzyme (0.5 U/μL) overnight at 50° C. and the digestion products were run on agarose gels. Imaging was done using the emission wavelengths corresponding to the two fluorophores, and after ethidium bromide staining, by UV visualization.

The resulting images are shown in FIGS. 1A and B for ATTO-488 and Cy3, respectively. It can be seen that increasing the percentage of dATP-ATTO-488 nucleotides resulted in oligonucleotides with higher fluorescence. However, the total amount of RCA product dropped by approximately 60% when 5% of the dATP nucleotides were dATP-ATTO-488.

Surprisingly and in contrast to the use of dATP-ATTO-488, the percentage of dATP-Cy3 nucleotides present did not seem to affect the efficiency of phi29 DNA polymerase; single stranded DNA products were visible with 5% of dATP-Cy3 in the RCA mixture (FIG. 1B). Moreover, incorporation of the modified nucleotides did not prevent or reduce the efficiency of the BtsCI restriction enzyme, and consequently the release of the designed hairpins comprising the cleavage domains.

SEQ ID NO: 1:
CCGGCGTCAATACGGGATAATACCGCGCCACATAGCAGAACTTTAAAAG
TGCTCATCATTGGAAAACGTTCTTCGGGGCGAAAACTCTCAAGGATCTT
ACCGCTGTTGAGATCCAGTTCGATGTAACCCACTCGTGCACCCAACTGA
TCTTCAGCATCTTTTACTTTCACCAGCGTTTCTGGGTGAGCAAAAACAG
GAAGGCAAAATGCCGCAAAAAAGGGAATAAGGGCGACACGGAAATGTTG
AATACTCATACTCTTCCTTTTTCAATATTATTGAAGCATTTATCAGGGT
TATTGTCTCATGAGCGGATACATATTTGAATGTATTTAGAAAAATAAAC
AAATAGGGGTTCCGCGCACATTTCCCCGAAAAGTG

### Example 2—Enzymatic Production of Single Stranded Oligonucleotides Comprising Nucleotides with Internalized Alkyne Groups, i.e. Alkyne Groups in the Nucleobase

Single stranded oligonucleotides 420 nucleotides in length (SEQ ID NO: 2) comprising nucleotides with alkyne groups were produced enzymatically using phi29 DNA polymerase.

A double stranded circular DNA template containing SEQ ID NO: 2 and hairpin cleavage domains was prepared as described in Ducani et al., 2013, Nature Methods, 647-652. Nicking of the template RCA reactions were performed using the conditions described in Example 1, but using increasing relative amounts of 5′-Ethynyl-dUTP (5′-EdUTP), i.e. replacing dTTP with 5′-EdUTP such that the relative amount of 5′-EdUTP was 0% (control reaction), 25%, 50%, 75% or 100%. After amplification, the RCA products were digested by the BtsCI restriction enzyme and loaded on to an agarose gel as described in Example 2.

The results in FIG. 2A surprisingly show that even when up 100% of the dTTP nucleotides were replaced with the alkyne functionalized dUTP, the RCA yields dropped only by 15-20%. Thus, FIG. 2A also demonstrates that the RCA product was successfully and efficiently cleaved by BtsCI. The incorporation of the alkyne functionalized dUTP nucleotide was confirmed by the fact that a lower mobility of the functionalized oligonucleotide was observed.

An additional experiment was performed replacing phi29 DNA polymerase with Bst DNA polymerase. Gel electrophoresis showed no changes in amplification yield up to 50% of functionalized dUTP, with the final product shifted compared the one with 25% of modified dUTP, confirming the incorporation of the modified nucleotide which has higher molecular weight than its corresponding natural nucleotide (dTTP) (FIG. 2B).

SEQ ID NO: 2:
ATTGAAGCATGCGGCGTGCATAATTCTCTTACTGTCATGCCATGCGTAA
GATACCACCACACCCGCATTCGCCATTCAGGCGGCCGCCACCGCGGTGG
AGCTCCAGCTGCTGTTTCCTGTGTAGAGTTGGTAGCTCTTGATCCGGTC
ATATTTGTTCCCTTTAGATCCGCCTCCATCTACAGGGCGCGTCCCCGCG
CTTAATGCGCGGCCTAACTACGGCTACACTAGAAGGACTTACCTTCGGA
AAAGAAATTGTTATCCGCTCACAAAAGCCAGAGTATTTAAGCTCCCTCG
TGCGCTCTCCTGTTCCGGGTTATTGTCTCATCGGCGACCGAGTTGCTCT
TGCTTATCAGACCCTGCCGCTTACAAGTGGTCGCCAGTCTATTAACAGC
ACTCAATACGGGATAATTTTTCAATATT

### Example 3—Click Chemistry Reaction to Conjugate Azide-Fluorophore to Single Stranded Nucleotide Comprising Internalized Alkyne Groups

The successful incorporation of the functionalized 5-Ethynyl-dUTP nucleotides into the oligonucleotide produced in Example 2 was further demonstrated by performing a click chemistry reaction.

The functionalized oligonucleotide from the reaction with 75% of the alkyne functionalized dUTP nucleotide was incubated with a Cy3-azide (50 μM). The click chemistry solution also included copper sulfate (50 μM) as a catalyst, sodium ascorbate (50 mM) and THPTA (250 μM). As negative control, an oligonucleotide produced by the same method from the same template but with conventional dNTPs was also incubated with the Cy3-azide. In addition, the functionalized oligonucleotide comprising internalized alkyne groups was also incubated in the absence of the Cy3-azide. The reaction mixtures from the three reactions were run on an agarose gel and imaged (FIG. 3). Fluorescent single stranded oligonucleotides of the expected length were observed only for the reaction comprising the functionalized oligonucleotide and the fluorophore-azide. In addition, no visible DNA degradation due to the presence of the copper sulfate was observed.

### Example 4—Enzymatic Production of Single Stranded Oligonucleotides Comprising Endonuclease Resistant Nucleotides

2′-Fluoro-2′-deoxyuridine-5′-triphosphate (2′F-dUTP) or 2′-Deoxythymidine-5′-O-(1-Thiotriphosphate) (phosphorothioate dTTP) have both been previously used to modify DNA and RNA oligonucleotides for biomedicine and therapeutics applications, due to their capacity of conferring nuclease stability. These modified nucleotides were incorporated into single stranded DNA oligonucleotides by RCA using the experimental schemes described above. The conventional dTTP nucleotides were replaced by the functionalized nucleotides in increasing percentages from 0 to 100%. The tandem repeat RCA products were then digested by the BtsCI restriction enzyme into discrete 420 base single stranded functionalized oligonucleotides (SEQ ID NO: 2).

In the experiment performed with the 2′F-dUTP functionalized nucleotides, similar RCA yields were visible from 0% up to 75% of the functionalized nucleotide, with a drastic drop in yield observed with 100% of the functionalized nucleotide (FIG. 4A).

In the phosphorothioate dTTP experiment, the RCA yield decreased gradually as the amount of the functionalized nucleotide was increased, up to approximately a 65% drop with 75% of the functionalized nucleotide, relative to the yield with only conventional nucleotides (FIG. 4B).

However, overexposure of the agarose gels showed how functionalized single stranded oligonucleotides were produced even with 100% of functionalized nucleobases—see the panels on the right of FIGS. 4A and B.

Other phosphorothioate dNTPs (indicated as Alpha S dNTPs) were tested in additional experiments, either added one by one or in combination (FIG. 4C). Even in the last case, in which 75% of all the conventional nucleotides were replaced with their corresponding Alpha S functionalized nucleotides, RCA products were synthesized and enzymatically cleaved to yield oligonucleotides visible on the agarose gel.

The endonuclease resistance of the functionalized oligonucleotides relative to control oligonucleotides produced with only conventional nucleotides was investigated. A control 420-nt long oligonucleotide produced with only conventional nucleotides, the 2′-F-dUTP functionalized oligonucleotides and phosphorothioate dTTP functionalized oligonucleotides (both produced using a relative amount of 75% of the functionalized nucleotide), were incubated with increasing concentrations of DNAse I (FIGS. 5A-C). The control oligonucleotide was completely digested with 18 mU/ml of DNAse I, but both the enzymatically produced 2′-F-dUTP and phosphorothioate dTTP functionalized DNA oligonucleotides were still visible on agarose gels after incubation with the same concentration of endonuclease.

### Example 5—Enzymatic Production of Single Stranded Oligonucleotides Comprising Nucleotides with Vinyl Groups

Single stranded DNA oligonucleotides with lengths from 76-81 bases (SEQ ID NOs: 3-13), functionalized with the thymidine analogue 5-Vinyl-2′-deoxyuridine-5′-triphosphate (5-Vinyl-dUTP) were produced enzymatically via an RCA reaction according to the experimental schemes described above. All of the oligonucleotides were encoded on a single pseudogene (SEQ ID NO: 16). The incorporation of such a functionalized nucleotide in single stranded oligonucleotides enables copper-free click chemistry reactions to be used to conjugate tetrazine-like molecules to the oligonucleotide. This alkene-tetrazine reaction can be completely orthogonal to the alkyne-azide click chemistry reaction previously performed.

Increasing the amount of the functionalized nucleotide in the RCA reaction mixture, relative to the conventional nucleotide dTTP, led to the successful incorporation of the 5-Vinyl-dUTP into the single stranded RCA product and the successful digestion of the hairpin structures. However, the activity levels of both the phi29 DNA polymerase and the type II endonuclease used to cleave the RCA product were lower than in the absence of the functionalized nucleotide, which consequently led to higher molecular weight bands with undigested hairpin structures when the functionalized nucleotides fully replaced the conventional dTTP nucleotides (FIG. 6).

| SEQ | |
|---|---|
| ID | |
| NO | Sequence |
| 3 | GAACCGTCCCAAGCGTTGCGCCACATCTGCTGGAAGGTGGAC |
| | AGTGAGAGGACACCTACGAATCGCAACGGGTATCCT |
| 4 | GAACCGTCCCAAGCGTTGCGCCTGGGTACATGGTGGTACCAC |
| | CAGACAGGACACCTACGAATCGCAACGGGTATCCT |
| 5 | GAACCGTCCCAAGCGTTGCGGAGAGCATAGCCCTCGTAGATG |
| | GGCAAGGACACCTACGAATCGCAACGGGTATCCT |
| 6 | GAACCGTCCCAAGCGTTGCGGTCCCAGTTGGTAACAATGCCA |
| | TGTTCAATGAGGACACCTACGAATCGCAACGGGTATCCT |
| 7 | GAACCGTCCCAAGCGTTGCGCGGACTCATCGTACTCCTGCTT |
| | GCTGAGGACACCTACGAATCGCAACGGGTATCCT |
| 8 | GAACCGTCCCAAGCGTTGCGTTCTCTTTGATGTCACGCACGAT |
| | TTCCCAGGACACCTACGAATCGCAACGGGTATCCT |
| 9 | GAACCGTCCCAAGCGTTGCGCTCGGTCAGGATCTTCATGAGG |
| | TAGTCTGTAGGACACCTACGAATCGCAACGGGTATCCT |
| 10 | GAACCGTCCCAAGCGTTGCGTTTCACGGTTGGCCTTAGGGTT |
| | CAGGGGAGGACACCTACGAATCGCAACGGGTATCCT |
| 11 | GAACCGTCCCAAGCGTTGCGGTACTTCAGGGTCAGGATACCT |
| | CTCTTGAGGACACCTACGAATCGCAACGGGTATCCT |
| 12 | GAACCGTCCCAAGCGTTGCGCTGCTCGAAGTCTAGAGCAACA |
| | TAGCACAAGGACACCTACGAATCGCAACGGGTATCCT |
| 13 | GAACCGTCCCAAGCGTTGCGCCTCGTCACCCACATAGGAGTC |
| | CTTCAGGACACCTACGAATCGCAACGGGTATCCT |

### Example 6—Enzymatic Production of Single Stranded Oligonucleotides Comprising Thiolated Nucleotides

Single stranded DNA oligonucleotides functionalized with a thiolated dTTP (4-Thiothymidine-5′-Triphosphate) were produced enzymatically via an RCA reaction using the reaction scheme and the templates described above (SEQ ID NOs: 3-13). All of the oligonucleotides were encoded on a single pseudogene (SEQ ID NO: 16)

The incorporation of the thiolated nucleotide into the single stranded oligonucleotide by phi29 DNA polymerase incorporation was very successful in amounts of the functionalized nucleotide up to 75% replacement of the corresponding conventional nucleotide (dTTP) (FIG. 7), i.e. a relative amount of 75% of the functionalized nucleotide.

The RCA products were digested by type II restriction enzymes as described above, however, a complete digestion of the functionalized RCA products required an enzyme concentration 10 times higher than the concentration used on RCA products comprising only conventional nucleotides. When the conventional dTTP nucleotide was completely replaced with the functionalized thiolated nucleotide, no single stranded oligonucleotides were observed following treatment with the type II restriction enzymes, though there only very faint accumulation of undigested RCA products was observed in the well, suggesting that the activity of the polymerase was also affected.

### Example 7—Enzymatic Production of Single Stranded Oligonucleotides Comprising Azide Nucleotides

Single stranded oligonucleotides 420 nucleotides in length (SEQ ID NO: 2) comprising increasing percentages of functionalized 2′-Azido-dATP nucleotides (FIG. 9A), which replace the corresponding conventional dATP nucleotides, were produced enzymatically using phi29 DNA polymerase (FIG. 9B) and Bst DNA polymerase (FIG. 9C).

The high density azido groups in the newly synthesized DNA strands enables the post-synthesis functionalization with alkyne molecules, either by a Cu(I)-catalyzed Huisgen cycloaddition (“click” chemistry), or by a strain-promoted [3+2] cycloaddition of azides and cycloalkynes, e.g. cyclooctyne or cyclononyne.

Two different polymerases with strand displacement activity were used in the amplification step: phi29 DNA polymerase or Bst DNA polymerase. Both polymerases were able to incorporate the functionalized nucleotide into the amplification products, which were then digested and run on an agarose gel.

DNA products produced with phi29 DNA polymerases were visible in the gel up to 75% of the modified nucleotides (FIG. 9B) while Bst DNA polymerases products were visible up to 100% (FIG. 9C), although Bst amplification buffer salts led to smear effect (even in the lane corresponding to 0% of 2′-Azido dATP). The functionalized nucleotides were incorporated successfully and did not significantly affect the formation of hairpin structures, which enable the cleavage of the amplification product.

### Example 8—Enzymatic Production of Single Stranded Oligonucleotides Comprising Biotinylated Nucleotides

Single stranded oligonucleotides 420 nucleotides in length (SEQ ID NO: 2) comprising increasing percentages (25%-100%) of functionalized Biotin-16-Aminoallyl-2′-dUTP (FIG. 10A), which replace the corresponding conventional nucleotide dTTP, were produced enzymatically using phi29 DNA polymerase (FIG. 10B).

The incorporation of the biotinylated nucleotides only slightly affected the DNA amplification reaction and, surprisingly, despite the potential for steric hindrance due to the large size of the functionalized nucleotide, did not interfere with the formation of the hairpin structures, which enable the cleavage of the RCA products. The incorporation of multiple internal biotins into a functionalized polynucleotide enables the conjugation with streptavidin functionalized molecules.

### Example 9—Enzymatic Production of Single Stranded Oligonucleotides Comprising 5-Modified Pyrimidines: 5-Bromo-2′-deoxyuridine-5′-Triphosphate and 5-Propynyl-2′-deoxycytidine-5′-Triphosphate

Single stranded oligonucleotides 420 nucleotides in length (SEQ ID NO: 2) comprising increasing percentages (25%-100%) of unnatural 5-modified pyrimidines: 5-Bromo-2′-deoxyuridine-5′-Triphosphate (FIG. 11A) and 5-Propynyl-2′-deoxycytidine-5′-Triphosphate (FIG. 11B), which replace the corresponding conventional nucleotides dTTP and dCTP, respectively, were produced enzymatically using phi29 DNA polymerase (FIGS. 11C and 11D) and Bst DNA polymerase (FIGS. 11E and 11F). Surprisingly, both functionalized nucleotides were successfully incorporated into the newly synthesized DNA sequence without affecting the formation of the hairpin structures in the RCA product, thus allowing the cleavage reaction to occur.

### Example 10—Enzymatic Production of Single Stranded Oligonucleotides Comprising 2′-O-Methyl-ATP

Single stranded oligonucleotides 420 nucleotides in length (SEQ ID NO: 2) comprising increasing percentages (25%-100%) of 2′-O-Methyl-ATP (FIG. 12A), which replace the corresponding conventional nucleotide dATP, were produced enzymatically using phi29 DNA polymerase (FIG. 12B). Even with 100% of the functionalized nucleotide present, the amplification product was still visible. This result was contrary to previous studies which showed that no known natural polymerases are capable of efficiently accepting these modified substrates (Romesberg, JACS 2004, 10.1021/ja038525p).

In addition, no higher molecular weight undigested DNA bands were visible in the gel, showing that the presence of the functionalized nucleotides did not affect the formation of the hairpin structure and its digestion by the restriction enzyme. This was a surprising result in view of the nuclease resistance which is conferred to DNA molecules by O-methyl groups.

### Example 11 Enzymatic Production of Single Stranded Oligonucleotides Comprising LNA-adenosine-5′-triphosphate

Single stranded oligonucleotides 420 nucleotides in length (SEQ ID NO: 2) comprising increasing percentages (25%-100%) of LNA-adenosine-5′-triphosphate (FIG. 13A), which replace the corresponding conventional nucleotide dATP, were produced enzymatically using phi29 DNA polymerase (FIG. 13B) and Bst DNA polymerase (FIG. 13C).

Although LNA monomers structurally mimic RNA, even with 100% of the functionalized nucleotide the efficiency of the polymerases, both phi29 DNA polymerase and Bst DNA polymerases, was not significantly affected. Moreover, the functionalized nucleotide did not interfere with the formation of the hairpin structures which enable digestion of the amplification products.

1. A method for producing single stranded functionalized oligonucleotides, said method comprising:
(a) providing a circular DNA molecule comprising an oligonucleotide sequence bordered by cleavage domains;
(b) performing a rolling circle amplification (RCA) reaction with the circular DNA molecule of (a) as a template and one or more functionalized nucleotides (dNTPs); and
(c) enzymatically cleaving the product of the RCA reaction at the cleavage domains to release the single stranded functionalized oligonucleotides.

2. The method of claim 1, wherein the circular DNA molecule is double stranded and wherein the method comprises an additional step of cleaving a single strand of the circular DNA molecule to provide an RCA template, before the RCA reaction is performed.

3. The method of claim 1, wherein the functionalized dNTPs are selected from the group consisting of: nucleotides comprising an alkyne group, fluorescently labeled nucleotides, nucleotides comprising a sterol group, nucleotides comprising a polyether group, nucleotides comprising a metal complex, nucleotides comprising a vinyl group, nucleotides comprising a thiol group, thionated nucleotides, nucleotides modified to have increased nuclease resistance, nucleotides comprising a chemical group capable of participating in a click chemistry reaction, and nucleotides that affect the thermostability of the oligonucleotide.

4. The method of any one of claim 1, wherein the functionalized dNTPs are nucleotides comprising an alkyne group, an alkene group, an azide group, a halogen group, an O-methyl group, a locked ribose sugar, preferably wherein the functionalized dNTPs are nucleotides comprising an alkyne group, a vinyl group or an azide group.

5. The method of claim 4, wherein the method further comprises a step of conjugating a molecule or component to the oligonucleotide via the alkyne, vinyl or azide group.

6. The method of claim 5, wherein the molecule or component is selected from the group consisting of: a fluorophore, a sterol, a polyether, a metal complex, molecule containing a thiol group, a molecule containing a group providing increased nuclease resistance and a molecule containing a group capable of participating in a click chemistry reaction.

7. The method of claim 1, wherein the cleavage domains (i) are directly adjacent to the oligonucleotide sequence; contain a sequence that is recognized by a cleavage enzyme; (iii) comprise or consist of a sequence capable of forming a hairpin structure; and/or (iv) that border the oligonucleotide sequence are the same.

8. (canceled)

9. (canceled)

10. The method of claim 7, wherein the double-stranded portion of the hairpin structure comprises a sequence that is recognized by a cleavage enzyme.

11. The method of claim 7, wherein the cleavage enzyme is a type II restriction endonuclease, optionally a type IIS restriction endonuclease, such as BseGI or BtsCI.

13. The method of claim 2, wherein the step of cleaving a single strand of the circular DNA molecule to provide an RCA template comprises cleaving a single strand of the circular DNA molecule with a cleavage enzyme.

14. The method of claim 13, wherein the circular DNA molecule contains a sequence that is recognized by the cleavage enzyme.

15. The method of claim 14, wherein the sequence that is recognized by the cleavage enzyme is between the cleavage domains that border the oligonucleotide sequence and is not in the oligonucleotide sequence.

16. The method of claim 13, wherein the cleavage enzyme is a nickase, optionally wherein the cleavage enzyme is Nb.BsrDI, Nt.BspQI or a combination thereof.

17. The method of claim 1, wherein the RCA reaction uses phi29 DNA polymerase or Bst DNA polymerase.

18. (canceled)

19. The method of claim 1, wherein the circular DNA molecule comprises a plurality of oligonucleotide sequences, wherein each oligonucleotide sequence is bordered by cleavage domains.

20. The method of claim 19, wherein the oligonucleotide sequences are different.

21. The method of claim 1, wherein step (a) comprises:
(i) cloning into a DNA plasmid a linear DNA molecule comprising the oligonucleotide sequence bordered by cleavage domains;
(ii) amplifying said plasmid;
(iii) excising part of the plasmid containing the DNA molecule comprising the oligonucleotide sequence bordered by cleavage domains; and
(iv) circularizing the part of the plasmid obtained in step (iii).

22. The method of claim 21, wherein step (ii) comprises transfecting said DNA plasmid into bacteria and growing the bacteria.

23. The method of claim 21, wherein the linear DNA molecule comprising the oligonucleotide sequence bordered by cleavage domains further comprises a 5′ end region and a 3′ end region each comprising a cleavage domain and wherein step (iii) comprises cleaving the cleavage domains in the end regions with a cleavage enzyme, optionally wherein said cleavage enzyme is BsmBI or BsaI.

24. The method of claim 1, further comprising a step of isolating or purifying the single stranded functionalized oligonucleotides.

25. (canceled)

26. (canceled)

27. A kit for use in the method of claim 1 comprising:
(i) a circular DNA molecule comprising an oligonucleotide sequence bordered by cleavage domains, wherein the cleavage domains comprise or consist of a sequence capable of forming a hairpin structure and wherein the double-stranded portion of the hairpin structure comprises a sequence that is recognized by a cleavage enzyme; and
(ii) functionalized dNTPs, optionally as defined in claim 3 or 4; and optionally
(iii) one or more cleavage enzymes that cleave the cleavage domains of (i).

28. The kit of claim 27, wherein the cleavage domains are as defined in claim 7 and/or the DNA molecule is as defined in claim 19.

29. A single stranded functionalized oligonucleotide obtained by the method of claim 1, wherein;
(i) the oligonucleotide contains at least 50 nucleotides; and
(ii) at least 5% of the nucleotide residues contain a functional group selected from an alkyne group, an alkene group, an azide group, a halogen group, an O-methyl group, a locked ribose sugar or a combination thereof.

30. The single stranded functionalized oligonucleotide of claim 29, wherein (i) at least one of the nucleotide residues containing a functional group is an internal residue; and/or (ii) at least 10% of the nucleotide residues contain a functional group selected from an alkyne group, an alkene group, an azide group, a halogen group, an O-methyl group, a locked ribose sugar or a combination thereof.

31. (canceled)

32. A library comprising a plurality of single stranded functionalized oligonucleotides obtained by the method of claim 1, wherein the library includes a single stranded functionalized oligonucleotide as defined in claim 29.

33. The method of claim 1, being a method for producing a pool of single stranded functionalized oligonucleotides for use in single molecule fluorescence in situ hybridization (smFISH), wherein the functionalized nucleotides are; (i) fluorescently labeled nucleotides and wherein each single stranded functionalized oligonucleotide in the pool contains about 15-30, preferably about 20-25, nucleotides; or (ii) nucleotides comprising a chemical group capable of participating in click chemistry, and wherein the method further comprises a step of conjugating a fluorescent label to at least one functionalized nucleotide in each functionalized oligonucleotide via click chemistry, and wherein each single stranded functionalized oligonucleotide in the pool contains about 15-30, preferably about 20-25, nucleotides.

34. (canceled)

35. The method of claim 33, wherein the nucleotides comprising a chemical group capable of participating in click chemistry are nucleotides comprising an azide group, an alkyne group, an alkene group, a nitrone group, a tetrazine group or a tetrazole group, or a combination thereof.

36. The method of claim 33, wherein the step of conjugating a fluorescent label to at least one functionalized nucleotide in each functionalized oligonucleotide via click chemistry is carried out before the functionalized oligonucleotides are hybridized to a nucleic acid molecule comprising a target sequence.

37. The method of claim 33, wherein the step of conjugating a fluorescent label to at least one functionalized nucleotide in each functionalized oligonucleotide via click chemistry is carried out after the functionalized oligonucleotides are hybridized to a nucleic acid molecule comprising a target sequence.
